(19) United States
(12) Patent Application Publication
(10) Pub. No.: US 2014/0163371 A1
(43) Pub. Date: Jun. 12, 2014

(54) TUMOR TISSUE REDUCTIVE PROCEDURE UNDER ULTRASOUND OBSERVATION

(71) Applicant: Olympus Medical Systems Corp., Tokyo (JP)

(72) Inventors: Shoichi MATSUI, Sagamihara-shi (JP); Kenichi NISHINA, Tokyo (JP); Kunihide KAJI, Tokyo (JP)

(73) Assignee: OLYMPUS MEDICAL SYSTEMS CORP., Tokyo (JP)

(21) Appl. No.: 14/100,332

(22) Filed: Dec. 9, 2013

Related U.S. Application Data
(60) Provisional application No. 61/736,228, filed on Dec. 12, 2012.

(51) Int. Cl. A61B 5/00; A61M 5/32; A61B 18/20; A61B 1/015; A61B 8/12; A61B 1/018; A61B 18/04; A61B 8/08; A61K 31/045

(52) U.S. Cl. CPC A61B 5/4839; A61B 8/085; A61M 5/32; A61B 18/20; A61K 31/045; A61B 8/12; A61B 1/018; A61B 18/04; A61B 1/015
USPC 600/439

(57) ABSTRACT

A tumor reductive procedure under ultrasound observation includes observing a tissue from an inside or an outside of a body with an ultrasound observation device, dissolving, breaking up, resecting, or vaporizing a tumor inner portion under ultrasound observation by the ultrasound observation device, introducing a solvent for suspension into the tumor inner portion from a distal end of a needle tube arranged in the tumor inner portion, mixing the solvent for suspension with a tumor tissue of the dissolved, broken-up, resected, or vaporized tumor inner portion, and changing the tumor inner portion to a fluid, and collecting the tumor tissue, which is mixed with the solvent for suspension and softened, from the tumor inner portion using the needle tube or another duct after changing the tumor to the fluid under the ultrasound observation.

### BACKGROUND OF THE INVENTION

1. Field of the Invention

The present invention relates to a tumor tissue reductive procedure for reducing a volume of a tumor under ultrasound observation.

2. Description of the Related Art

Conventionally, for complete curing of a disease involving a lesion part such as a tumor, a surgical operation for excising the lesion part is preferable. However, in a state in which an organ, in particular, a blood vessel around the tumor is compressed or infiltrated according to growth and progress of the tumor, in some case, various clinical conditions such as a pain or dysfunction of an organ occur and the surgical operation cannot be performed. For example, since pancreatic cancer is cancer that occurs in a relatively small pancreas, when the pancreatic cancer increases in volume, the pancreatic cancer easily involves compression and infiltration to surrounding organs such as a bile duct and a duodenum, nerves, and blood vessels. Therefore, the pancreatic cancer is considered a refractory disease for which application of the surgical operation is limited and treatment is difficult.

As a therapy other than the surgical operation for a lesion part such as a tumor, in general, there are a chemotherapy and a radiation therapy. In recent years, as disclosed in Japanese Patent Application Laid-Open Publication No. 2010-533718, a therapy using a virus is also proposed.

### SUMMARY OF THE INVENTION

A tumor reductive procedure under ultrasound observation according to an aspect of the present invention includes: observing a tissue from an inside or an outside of a body with an ultrasound observation device; dissolving, breaking up, resecting, or vaporizing a tumor inner portion under ultrasound observation by the ultrasound observation device; introducing a solvent for suspension into the tumor inner portion from a distal end of a needle tube arranged in the tumor inner portion, mixing the solvent for suspension with a tumor tissue of the dissolved, broken-up, resected, or vaporized tumor inner portion, and changing the tumor inner portion to a fluid; and collecting the tumor tissue, which is mixed with the solvent for suspension and softened, from the tumor inner portion using the needle tube or another duct after changing the tumor inner portion to the fluid.

### BRIEF DESCRIPTION OF THE DRAWINGS

FIG. 1 is a configuration diagram showing a configuration of an ultrasound endoscope apparatus according to a first embodiment of the present invention.

FIG. 2 is a schematic explanatory diagram of a human body including a pancreas and a stomach.

FIG. 3 is a diagram for explaining a flow of a manipulation for reducing a volume of a tumor part Pa, which is a tumor part of a pancreas P, according to the first embodiment of the present invention.

FIG. 4 is a diagram for explaining a projecting state of a needle tube distal end portion 22a according to the first embodiment of the present invention.

FIG. 5 is a diagram showing an example of an ultrasound image displayed on a monitor 4 according to the first embodiment of the present invention.

FIG. 6 is a diagram for explaining a state at the time when trypsin is injected into the tumor part Pa according to the first embodiment of the present invention.

FIG. 7 is a diagram showing an example of an ultrasound image displayed on the monitor 4 in the state shown in FIG. 6.

FIG. 8 is a diagram for explaining a state at the time when a dissolved tumor tissue in the tumor part Pa and ethanol are sucked according to the first embodiment of the present invention.

FIG. 9 is a diagram showing an example of an ultrasound image displayed on the monitor 4 in the state shown in FIG. 8.

FIG. 10 is a diagram for explaining a situation in which reflux of the ethanol is performed using two needle tubes 22 according to a modification 1 of the first embodiment of the present invention.

FIG. 11 is a partial sectional view of the needle tube distal end portion 22a through which a laser probe is inserted in order to warm a substance for dissolving a tumor tissue according to a modification 2 of the first embodiment of the present invention.

FIG. 12 is a partial sectional view of the needle tube distal end portion 22a through which a heat probe is inserted in order to warm a tumor region according to the modification 2 of the first embodiment of the present invention.

FIG. 13 is a diagram for explaining a procedure of a manipulation in a second embodiment of the present invention.

FIG. 14 is a schematic sectional view showing a configuration of an ultrasound probe inserted through a needle tube distal end portion for breaking up a tumor tissue with an ultrasound vibration according to a third embodiment of the present invention.

FIG. 15 is a configuration diagram showing a configuration of an ultrasound endoscope apparatus 1A according to a third embodiment of the present invention.

FIG. 16 is a diagram for explaining a procedure of a manipulation in the third embodiment.

FIG. 17 is a schematic sectional view showing a configuration of a modification of the ultrasound probe inserted through the needle tube distal end portion for breaking up a tumor tissue with an ultrasound vibration according to the third embodiment of the present invention.

FIG. 18 is a configuration diagram showing a configuration of an ultrasound endoscope apparatus 1B according to a fourth embodiment of the present invention.

FIG. 19 is a plan view of a distal end portion of a puncture tool insertion portion 79A according to the fourth embodiment of the present invention.

FIG. 20 is a sectional view of the distal end portion of the puncture tool insertion portion 79A taken along line XX-XX in FIG. 19.

FIG. 21 is a sectional view of the distal end portion of the puncture tool insertion portion 79A taken along line XXI-XXI in FIG. 19.

FIG. 22 is a perspective view of a distal end portion of a blade 72 according to the fourth embodiment of the present invention.

FIG. 23 is an external view of a puncture tool operation portion 79B according to the fourth embodiment of the present invention.

FIG. 24 is a sectional view of the puncture tool operation portion 79B taken along line XXIV-XXIV in FIG. 23.

FIG. 25 is a sectional view of the puncture tool operation portion 79B in a state in which a sheath 31 projects most from a treatment instrument opening of a distal end rigid portion 15 according to the fourth embodiment of the present invention.

FIG. 26 is a sectional view of the puncture tool operation portion 79B in a state in which the sheath 31 and a needle tube 71 project most from the treatment instrument opening of the distal end rigid portion 15 according to the fourth embodiment of the present invention.

FIG. 27 is a sectional view of the puncture tool operation portion 79B in a state in which the sheath 31 and the needle tube 71 project most from the treatment instrument opening of the distal end rigid portion 15 and the blade 72 is projected by one stage from the treatment instrument opening of the distal end rigid portion 15 according to the fourth embodiment of the present invention.

FIG. 28 is a sectional view of the puncture tool operation portion 79B in a state in which the sheath 31 and the needle tube 71 project most from the treatment instrument opening of the distal end rigid portion 15 and the blade 72 is projected most from the treatment instrument opening of the distal end rigid portion 15 according to the fourth embodiment of the present invention.

FIG. 29 is a sectional view showing a state in which a blade distal end portion 72a is in contact with a distal end portion 71d1 of an internal space 71d but the blade distal end portion 72a is not bent according to the fourth embodiment of the present invention.

FIG. 30 is a sectional view showing a state in which the blade distal end portion 72a is in contact with the distal end portion 71d1 of the internal space 71d, the blade 72 is further pushed out to a distal end side, and the blade distal end portion 72a is bent according to the fourth embodiment of the present invention.

FIG. 31 is a diagram for explaining a procedure of a manipulation in the fourth embodiment.

FIG. 32 is a diagram for explaining a projecting state of the blade distal end portion 72a according to the fourth embodiment of the present invention.

FIG. 33 is a diagram showing an example of an ultrasound image displayed on the monitor 4 according to the fourth embodiment of the present invention.

FIG. 34 is a diagram for explaining a movement of the blade distal end portion 72a according to the fourth embodiment of the present invention.

FIG. 35 is a diagram for explaining injection of saline or ethanol according to the fourth embodiment of the present invention.

FIG. 36 is a diagram showing an example of an ultrasound image displayed on the monitor 4 when the saline or the ethanol is injected according to the fourth embodiment of the present invention.

FIG. 37 is a diagram for explaining collection of resected tumor tissue pieces according to the fourth embodiment of the present invention.

FIG. 38 is a diagram showing an example of an ultrasound image displayed on the monitor 4 when the resected tumor tissue pieces are collected according to the fourth embodiment of the present invention.

FIG. 39 is a configuration diagram showing a configuration of an ultrasound endoscope apparatus according to a fifth embodiment of the present invention.

FIG. 40 is a schematic sectional view showing a configuration of a needle tube distal end portion through which a laser catheter 93 for radiating laser light on a tumor tissue and vaporizing the tumor tissue is inserted according to the fifth embodiment of the present invention.

FIG. 41 is a diagram for explaining a procedure of a manipulation in the fifth embodiment.

FIG. 42 is a diagram for explaining a projecting state of the needle tube distal end portion 22a of the needle tube 22 according to the fifth embodiment of the present invention.

FIG. 43 is a diagram showing an example of an ultrasound image displayed on the monitor 4 corresponding to FIG. 42.

FIG. 44 is a diagram for explaining a state at the time when laser light is radiated on a tumor tissue and the tumor tissue is vaporized according to the fifth embodiment of the present invention.

FIG. 45 is a diagram showing an example of an ultrasound image displayed on the monitor 4 in the state shown in FIG. 44.

FIG. 46 is a diagram for explaining a state at the time when saline or ethanol is injected into the tumor part Pa according to the fifth embodiment of the present invention.

FIG. 47 is a diagram showing an example of an ultrasound image displayed on the monitor 4 in the state shown in FIG. 46.

FIG. 48 is a flowchart showing a flow of a manipulation including processing for dissemination prevention in the first embodiment according to a modification 10.

FIG. 49 is a flowchart showing a flow of another manipulation including processing for dissemination prevention in the first embodiment according to the modification 10.

FIG. 50 is a diagram for explaining a method of discharging a tumor, which is mixed with a solvent for suspension and softened, to a stomach S, which is a digestive tract, from an inner portion of a tumor part using an internal biliary drainage tube 101 according to a modification 11.

FIG. 51 is a diagram for explaining a method of discharging a tumor, which is mixed with a solvent for suspension and softened, to an outside of a body from an inner portion of a tumor part using an external biliary drainage tube 102 according to the modification 11.

### DETAILED DESCRIPTION OF PREFERRED EMBODIMENTS

Embodiments of the present invention are explained below with reference to the drawings.

Note that, in the respective drawings used for the following explanation, a scale is varied for each of components in order to show respective components in volumes enough for being recognized on the drawings. The present invention is not limited to only numbers of the components shown in the drawings, shapes of the components, ratios of volumes of the components, and relative positional relations among the respective components.

### First Embodiment

### Apparatus Configuration

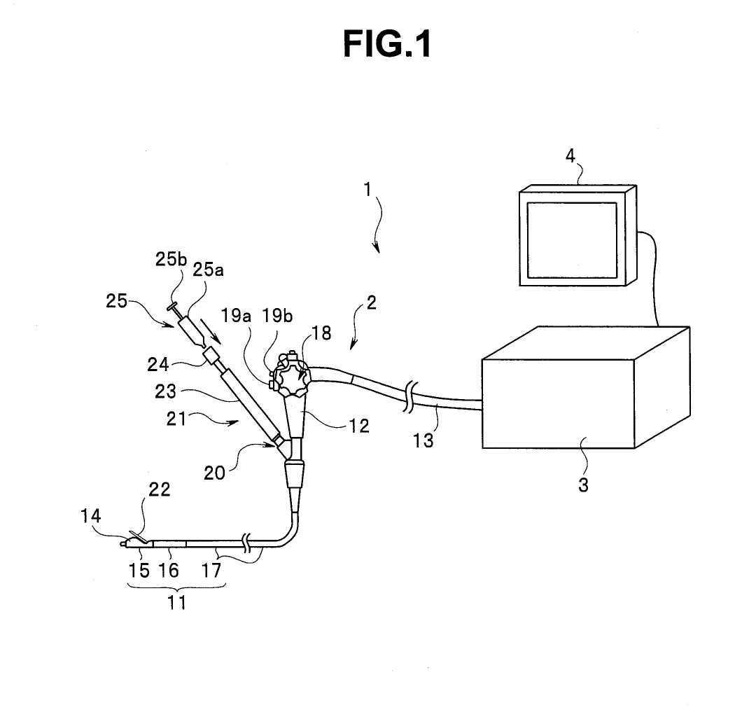

First, a configuration of an apparatus used for performing a tumor tissue reductive procedure under ultrasound observation according to the present embodiment is explained on the basis of FIG. 1. FIG. 1 is a configuration diagram of a configuration of an ultrasound endoscope apparatus according to the present embodiment.

As shown in FIG. 1, an ultrasound endoscope apparatus 1 is configured mainly by an ultrasound endoscope 2, an ultrasound observation device 3, and a monitor 4.

The ultrasound endoscope (hereinafter simply referred to as endoscope as well) 2 includes an endoscope insertion portion (hereinafter simply referred to as insertion portion as well) 11, an endoscope operation portion (hereinafter simply referred to as operation portion as well) 12, and a universal cord 13. An ultrasound probe 14 including an ultrasound probe is provided on a distal end side of the insertion portion 11. The ultrasound probe 14 includes a convex type transducer. An ultrasound connector (not shown in the figure) connected to the ultrasound observation device 3 is provided at a proximal end portion of the universal cord 13. The universal cord 13 extends from a side of the operation portion 12.

The insertion portion 11 having flexibility is configured by consecutively connecting a distal end rigid portion 15, a bendable bending portion 16, and a small-diameter elongated flexible tube portion 17 having flexibility. The operation portion 12 is disposed on a proximal end side of the insertion portion 11. An angle knob 18 for bending the bending portion 16 in a desired direction, a water feeding button 19a for performing water feeding operation, a suction button 19b for performing suction operation, and the like are provided in the operation portion 12.

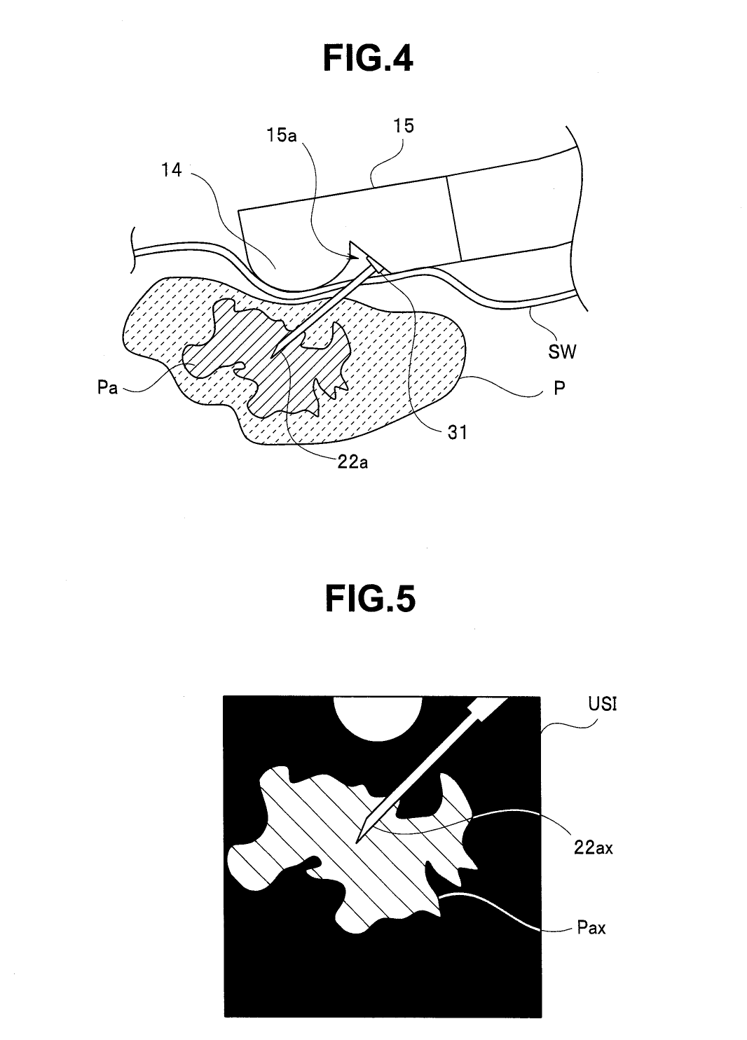

A treatment instrument insert-through port 20 is provided on a distal end portion side of the operation portion 12. A puncture device 21, which is a medical instrument for performing treatment, can be attached to the treatment instrument insert-through port 20. A distal end portion (hereinafter referred to as needle tube distal end portion) 22a of a needle tube 22 of the puncture device 21 is projected from a distal end opening portion 15a (FIG. 4), which is provided in the distal end rigid portion 15, through an instrument channel (not shown in the figure) in the insertion portion 11.

The puncture device 21 includes a main body portion 23, an operation slider 24, and the needle tube 22, a proximal end portion of which is connected to the operation slider 24. A doctor can project the needle tube distal end portion 22a from the distal end opening portion 15a of the distal end rigid portion 15 by operating the operation slider 24. The needle tube 22 is formed of stainless steel, a cobalt chrome alloy, a shape memory alloy (a nickel titanium alloy, etc.), or the like. An outer diameter of the needle tube 22 is 25 G to 19 G (gauge).

A syringe 25 can be mounted on a proximal end portion of the operation slider 24. A plunger 25b, which is inserted through a syringe main body 25a of the syringe 25, is pushed into the syringe main body 25a, whereby liquid in the syringe main body 25a can be injected into the needle tube 22. The plunger 25b is pulled out from the syringe main body 25a, whereby the liquid in the needle tube 22 can be sucked.

Note that the needle tube 22 is inserted through a sheath 31 (FIG. 4).

### (Manipulation)

An example of a manipulation for reducing a volume of a tumor of pancreatic cancer is explained. The manipulation in the present embodiment is a tumor tissue dissolving procedure and, specifically, a manipulation that makes use of an ultrasound endoscopic guided fine needle injection (EUS-FNI).

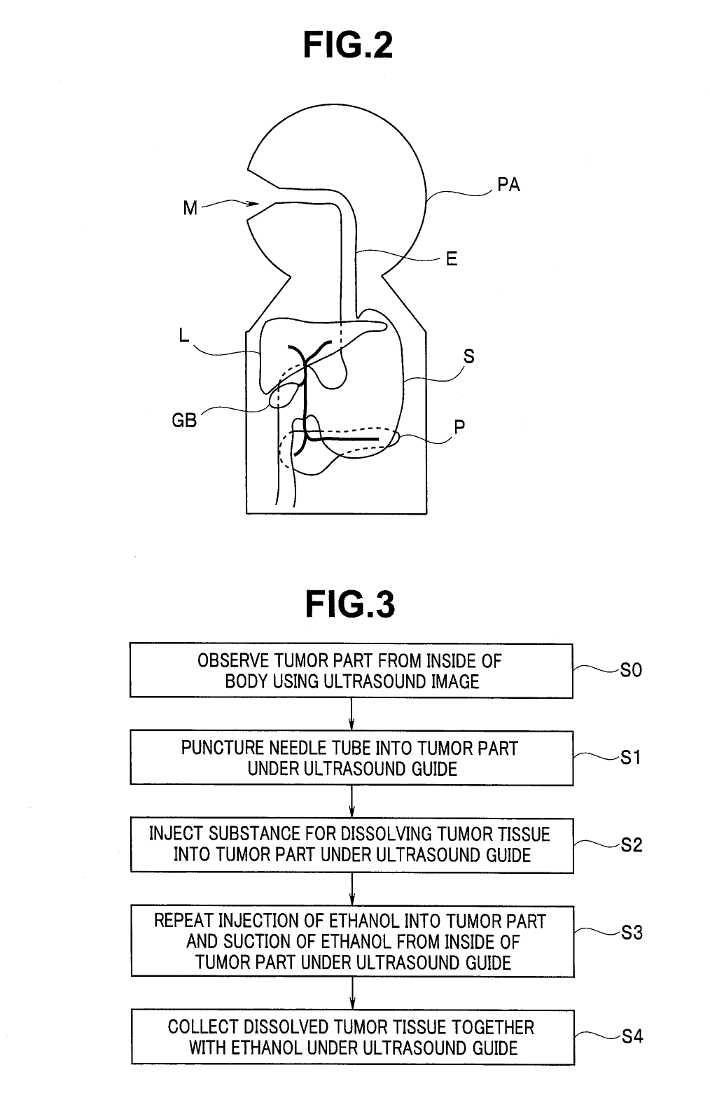

FIG. 2 is a schematic explanatory diagram of a human body including a pancreas and a stomach. The doctor inserts the insertion portion 11 of the endoscope 2 from a mouth M of the human body to a stomach S through an esophagus E. A pancreas P is present in a vicinity of the stomach S. A liver L and a gallbladder GB are present around the stomach S.

FIG. 3 is a diagram for explaining a flow of a manipulation for reducing a volume of a tumor part Pa, which is a lesion part of the pancreas P. First, as shown in FIG. 2, the doctor inserts a distal end portion of the insertion portion 11 from the mouth M of a patient PA through the esophagus E.

The doctor brings the ultrasound probe 14 of the distal end portion of the insertion portion 11 into close contact with a stomach wall of the stomach S in a vicinity of the tumor part Pa, performs ultrasound scanning, and observes the tumor part Pa of the pancreas P, which is a target of observation, from an inside of a body using an ultrasound image of the ultrasound observation device (S0). Subsequently, the doctor punctures the needle tube 22 into the tumor part Pa under an ultrasound guide (S1). That is, the doctor operates the operation slider 24 while viewing an ultrasound image displayed on the monitor 4, projects the needle tube distal end portion 22a of the needle tube 22, and punctures the needle tube 22 into the tumor part Pa of the pancreas P.

FIG. 4 is a diagram for explaining a projecting state of the needle tube distal end portion 22a. In FIG. 4, the needle tube distal end portion 22a inserted through the sheath 31 pierces through a stomach wall SW and enters into the tumor part Pa of the pancreas P. A position of the needle tube distal end portion 22a with respect to the tumor part Pa can be adjusted by the operation slider 24.

Note that the manipulation is performed through the stomach wall SW. However, the manipulation may be performed through an intestinal wall of a duodenum.

FIG. 5 is a diagram showing an example of the ultrasound image displayed on the monitor 4. An ultrasound image USI obtained by the ultrasound probe 14 is displayed on a display screen of the monitor 4. An ultrasound reflection machining portion is applied to the needle tube distal end portion 22a. In the ultrasound image USI, an image 22ax of the needle tube distal end portion 22a is clearly displayed. In the ultrasound image USI, an image Pax of the tumor part Pa is also displayed. Therefore, the doctor can observe the tumor from the inside of the body using the ultrasound observation device 3.

Therefore, the doctor drives the ultrasound probe 14 and adjusts, while viewing an ultrasound tomogram of the stomach wall SW and an organ present behind the stomach wall SW displayed on the monitor 4, a position of the distal end rigid portion 15 and a bending amount of the bending portion 16 such that the tumor part Pa is located in a middle of the ultrasound tomogram. Therefore, the doctor can locate, while viewing the ultrasound image USI, the distal end rigid portion 15 of the insertion portion 11 in a position where the needle tube distal end portion 22a can be punctured into the tumor part Pa of the pancreas P where the needle tube distal end portion 22a projects from the distal end opening portion 15a provided in the distal end rigid portion 15.

Subsequently, the doctor injects a substance for dissolving a tumor tissue into the tumor part Pa under the ultrasound guide (S2). That is, the doctor dissolves a tumor inner portion by injecting the substance for dissolving a tumor tissue under ultrasound observation by the ultrasound observation device 3.

Specifically, under the ultrasound observation, a distal end of the needle tube 22 is introduced into the tumor inner portion and the substance for dissolving a tumor inner portion is injected through the needle tube 22, whereby the tumor inner portion is dissolved.

The substance for dissolving a tumor tissue is poured into the syringe main body 25a in advance. The substance for dissolving a tumor tissue is, for example, trypsin, which is a digestive enzyme. In an example explained below, the trypsin is explained as one of substances for dissolving a tumor tissue. The doctor can adjust an injection amount of the trypsin while viewing the ultrasound image USI. The trypsin, which is the substance for dissolving a tumor tissue, separates or loosens bonding of cells or tissues.

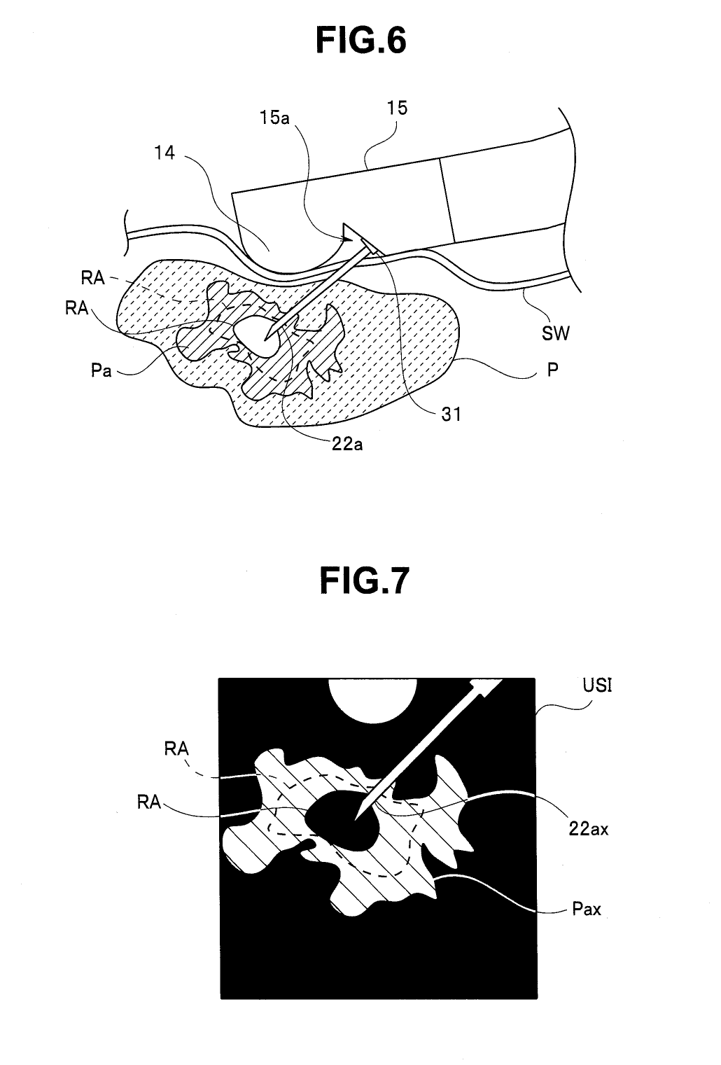

FIG. 6 is a diagram for explaining a state at the time when the trypsin is injected into the tumor part Pa. FIG. 7 is a diagram showing an example of an ultrasound image displayed on the monitor 4 in the state shown in FIG. 6.

When the trypsin is injected into the tumor part Pa, the trypsin dissolves a tumor tissue. The tumor tissue is decomposed by the trypsin to dissolve. FIG. 6 shows that the trypsin injected by the syringe 25 spreads to a region RA of the tumor part Pa. The region RA indicates a region into which the trypsin is injected. In the ultrasound image USI shown in FIG. 7, a portion of the region RA into which the trypsin is injected is rendered in black.

The doctor injects the trypsin dividedly several times while viewing a state of the dissolution of the tumor tissue in the tumor part Pa by the trypsin using the ultrasound image USI displayed on the monitor 4. For example, the trypsin is injected into the tumor part Pa at an interval of several seconds.

When the trypsin is injected by a predetermined amount, for example, 0.5 to 1.0 ml, the tumor tissue coming into contact with liquid of the trypsin pushed out from the needle tube distal end portion 22a dissolves. In the ultrasound image USI, a dissolved portion of the tumor tissue around the needle tube distal end portion 22a changes to a black image. Therefore, the doctor can check a range of the dissolved tumor tissue using the trypsin injected into the tumor tissue.

Subsequently, the doctor performs injection of the predetermined amount of the trypsin in order to dissolve the tumor tissue not dissolved yet while viewing the ultrasound image.

The trypsin is additionally injected, whereby the region RA expands to be large as indicated by dotted lines in FIGS. 6 and 7. For example, the doctor injects the trypsin dividedly several times at an interval of several seconds while viewing an ultrasound image displayed on the monitor 4.

The doctor repeats the manipulation until the entire tumor tissue dissolves. For example, when the doctor dissolves the entire tumor tissue, the doctor dissolves the tumor tissue to leave only a layer portion on an outer side surface of the tumor tissue. The doctor can determine, by viewing the ultrasound image, whether the tumor tissue is dissolved leaving only the layer portion on the outer side surface of the tumor tissue.

Only the layer portion on the outer surface side of the tumor tissue is left in order not to dissolve even a normal tissue. Note that a region to be dissolved may be only a desired region of the tumor part Pa rather than the entire tumor tissue.

As explained above, the doctor injects the trypsin dividedly several times while viewing the ultrasound image until the desired region in the tumor part Pa is dissolved. An injection amount and a number of times of injection of the trypsin are adjusted according to a volume of the tumor part Pa.

When the trypsin is injected into the desired region and the desired region is dissolved, the doctor pulls out a plunger of the syringe 25 to suck the dissolved tumor tissue and the remaining trypsin.

Subsequently, under the ultrasound guide, after injecting a trypsin inhibitor into the tumor part Pa according to necessary to surely suppress action of the remaining trypsin, the doctor repeats injection of ethanol and suction of the ethanol from the inside of the tumor part Pa (S3).

The doctor replaces the syringe 25 with a syringe into which the ethanol, which is a solvent for suspension, is poured and pushes in the plunger 25b of the syringe 25 to inject the ethanol into the region RA in which the tumor tissue in the tumor part Pa is dissolved. Further, the doctor pulls out the plunger 25b of the syringe 25 to suck the injected ethanol.

The doctor repeats the injection of the ethanol into the tumor part Pa and the suction of the ethanol from the inside of the tumor part Pa several times while viewing the ultrasound image.

As explained above, under the ultrasound observation, the doctor introduces the solvent for suspension into a tumor inner portion from the distal end of the needle tube 22 arranged in the tumor inner portion, mixes the solvent for suspension with the dissolved tumor tissue in the tumor inner portion, and changes the tumor inner portion to fluid.

Subsequently, the doctor collects the dissolved tumor tissue together with the ethanol under the ultrasound guide (S4).

That is, after changing the tumor inner portion to the fluid, the doctor collects the tumor tissue, which is mixed with the solvent for suspension and softened, from the tumor inner portion using the needle tube 22. Specifically, the doctor pulls out the plunger of the syringe 25 to suck and collect the dissolved tumor tissue and injected ethanol while viewing the ultrasound image.

When the dissolved tumor tissue and the ethanol are sucked according to S4, since a portion where the dissolved tumor tissue is present disappears, the volume of the tumor part Pa decreases.

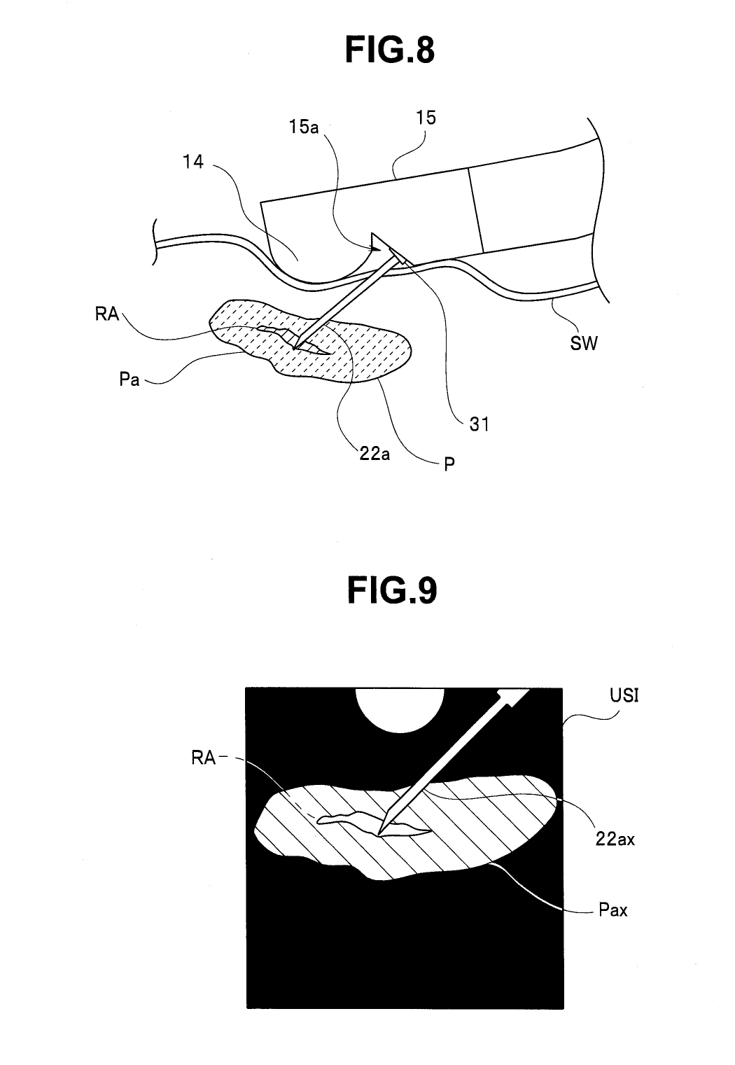

FIG. 8 is a diagram for explaining a state at the time when the dissolved tumor tissue in the tumor part Pa and the ethanol are sucked. FIG. 9 is a diagram showing an example of an ultrasound image displayed on the monitor 4 in the state shown in FIG. 8.

As shown in FIG. 8, since a part of the tumor part Pa is dissolved and sucked, the volume of the tumor part Pa decreases. As shown in FIG. 9, the image Pax of the tumor part Pa also decreases in volume.

Therefore, it is possible to reduce the volume of the tumor and reduce or eliminate compression of surrounding organs, blood vessels, and nerves. It is possible to perform a surgical operation such as excision of the tumor. Further, since the tumor tissue is sucked together with the ethanol after being dissolved, there is also an effect that tumor lysis syndromes due to remaining of a cancer cell or the like in the body are suppressed. A pain due to compression of nerves based on an increase in a tumor volume is also reduced and QOL (quality of life) is also improved.

Next, modifications of the present embodiment are explained.

### (Modification 1)

In the example explained above, in S3, the doctor repeats the injection and the suction of the ethanol using the syringe 25. However, the doctor may use two needle tubes, use one needle tube for the injection of the ethanol, and use the other needle tube for the suction of the ethanol to reflux the ethanol. Further fluidization of the tumor inner portion is performed by the refluxing.

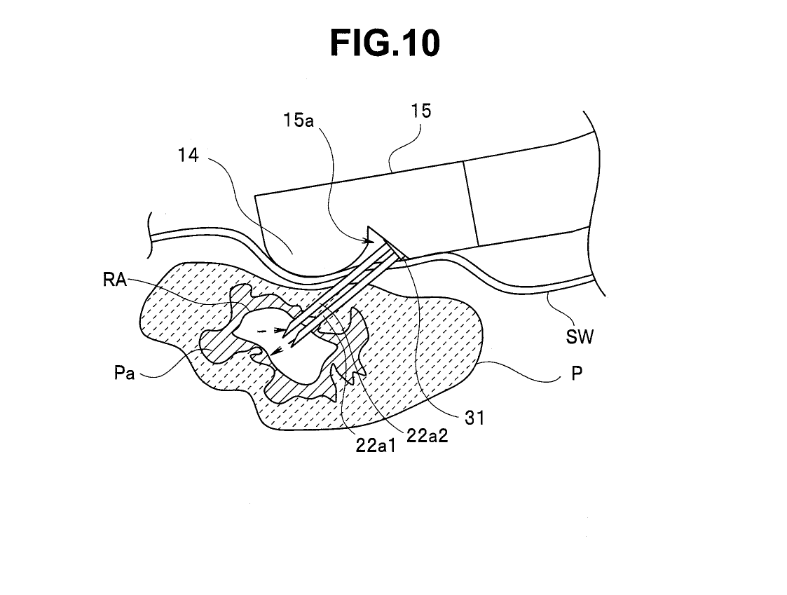

FIG. 10 is a diagram for explaining a situation in which the reflux of the ethanol is performed using two needle tubes 22. The two needle tubes 22 are inserted through the sheath 31 of the puncture device 21. As shown in FIG. 10, two needle tube distal end portions 22a1 and 22a2 of the two needle tubes 22 project from the distal end opening portion 15a and are punctured into the tumor part Pa.

In S2, the doctor performs the injection of the trypsin using one needle tube of the two needle tubes 22 and then sucks the trypsin using the other needle tube.

Specifically, as indicated by a dotted line in FIG. 10, in S3, the doctor performs the reflux of the ethanol by performing the suction of the ethanol via the other needle tube distal end portion 22a2 while performing the injection of the ethanol via one needle tube distal end portion 22a1. A pump for performing the supply of the ethanol is connected to the needle tube 22a1. A pump for performing the suction of the ethanol is connected to the needle tube 22a2.

That is, the processing in S3 can also be performed by puncturing such two needle tubes into the region RA where the tumor tissue dissolves and performing the reflux of the ethanol.

Note that, after S2, the doctor may pull out the needle tube 22 and insert a double lumen tube into the sheath 31 instead of the needle tube 22 to perform the reflux of the ethanol in the same manner as the reflux of the ethanol performed when the two needle tubes are punctured.

The doctor can determine a situation of the refluxing viewing the ultrasound image USI.

### (Modification 2)

In this modification, in order to improve activation of the substance for dissolving a tumor tissue, the substance for dissolving a tumor tissue is warmed to a predetermined temperature. In S2, after injecting the substance for dissolving a tumor tissue into the tumor part Pa, the doctor inserts a laser probe or a heat probe through the needle tube 22 to warm the substance for dissolving a tumor tissue.

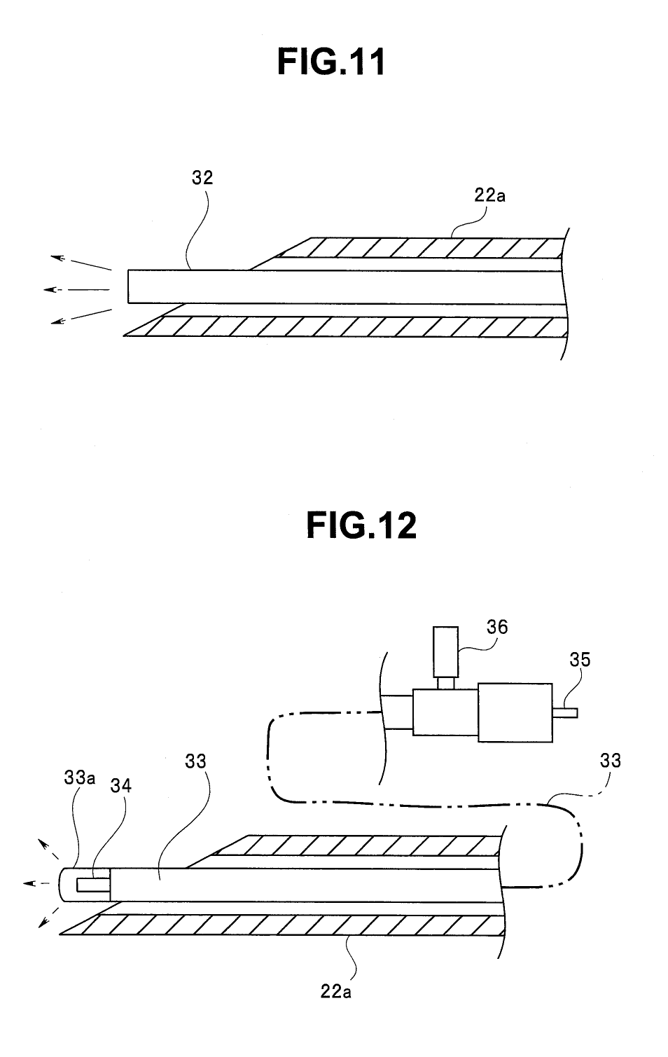

FIG. 11 is a partial sectional view of the needle tube distal end portion 22a through which the laser probe is inserted in order to warm the substance for dissolving a tumor tissue. A laser probe 32 is inserted through the needle tube 22. A distal end portion of the laser probe 32 projects from the needle tube distal end portion 22a. Laser light from a laser light source device (not shown in the figure) connected to a proximal end of the laser probe 32 is emitted from the distal end portion of the laser probe 32. According to the emission of the laser light, the trypsin is heated and activity of the trypsin is improved. An emitted light amount and an emission time of the laser light necessary for temperature of a probe distal end of the laser probe 32 to reach a predetermined temperature, for example, 42 degrees are calculated in advance by an experiment or the like. The laser light is outputted at the predetermined emitted light amount for the predetermined emission time. Note that, since a tumoricidal effect can be expected by heating the tumor tissue to temperature equal to or higher than 40 degrees, it is possible to improve a therapeutic effect.

FIG. 12 is a partial sectional view of the needle tube distal end portion 22a through which the heat probe is inserted in order to warm a tumor region. A heat probe 33 is inserted through the needle tube 22. A light emitting diode (hereinafter abbreviated as LED) 33a functioning as a heating element is provided at a distal end portion of the heat probe 33. The heat probe 33 includes a water feeding channel. A water feeding nozzle 34 communicating with the water feeding channel is provided at the distal end portion of the heat probe 33.

A cap for water feeding 35 and an electric connector 36 for the LED 33a are provided at a proximal end portion of the heat probe 33. A water feeding tube (not shown in the figure) from a water feeding pump (not shown in the figure) is connected to the cap for water feeding 35. A cable (not shown in the figure) from a power supply (not shown in the figure) for the LED 33a is connected to the electric connector 36.

The distal end portion of the heat probe 33 projects from the needle tube distal end portion 22a. Light from the LED 33a provided at a proximal end of the heat probe 33 is emitted from the distal end portion of the heat probe 33. Note that, at the same time, since water spouts from the water feeding nozzle 34, it is possible to prevent heat of the distal end portion of the heat probe 33 from being conducted to the proximal end side of the heat probe 33. Therefore, according to the emission of the light from the LED 33a, the trypsin is heated and activity of the trypsin is improved. An output light amount and an output time of the LED 33a necessary for setting temperature of a probe distal end of the heat probe 33 to a predetermined temperature, for example, 42 degrees for damaging only the tumor tissue without affecting a normal tissue are calculated in advance by an experiment or the like. The LED 33a is driven at the predetermined output light amount for the predetermined output time.

Thereafter, the laser probe 32 or the heat probe 33 is pulled out from the needle tube 22 and the processing in S3 is performed.

Note that, in the example explained above, the laser probe 32 or the heat probe 33 is inserted through the needle tube 22. However, the doctor may pull out the needle tube 22 and directly insert the laser probe 32 or the heat probe 33 through the sheath 31.

Furthermore, the ultrasound probe 14 provided on the distal end side of the insertion portion 11 may be ultrasonically vibrated at a predetermined frequency for a predetermined time to heat the ultrasound probe 14 to a predetermined temperature, for example, 42° C. to thereby warm the substance for dissolving a tumor tissue.

### Second Embodiment

In the first embodiment, the substance for dissolving a tumor tissue is injected into the tumor tissue and the dissolved tumor tissue is collected. However, in a second embodiment, when fibrillization of a tumor tissue advances, a substance for dissolving a tissue of the fiber is injected into the tumor tissue and the dissolved tumor tissue is collected.

An apparatus configuration for realizing a manipulation in the present embodiment is the same as the apparatus configuration in the first embodiment.

In the manipulation in the second embodiment, steps of a procedure same as in the first embodiment are denoted by the same reference signs and explanation of the steps is omitted.

### (Manipulation)

The manipulation in the second embodiment is also the tumor tissue dissolving procedure. Specifically, the manipulation is a manipulation that makes use of the ultrasound endoscopic chemical injection method (EUS-FNI).

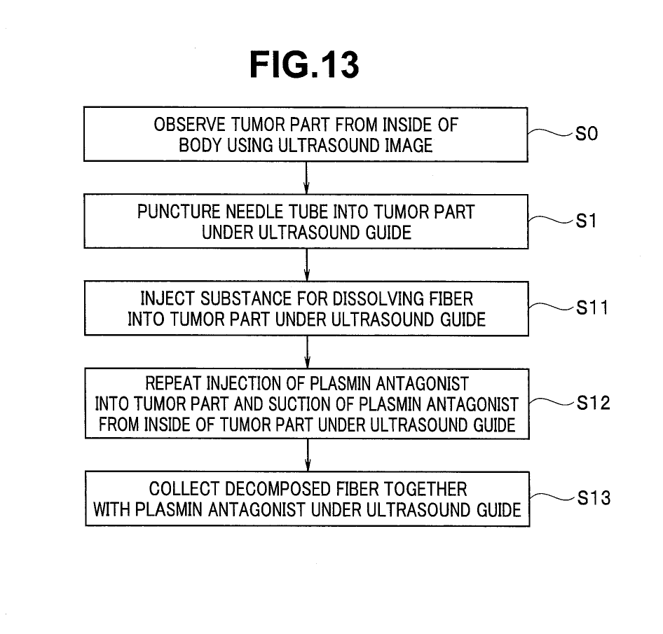

FIG. 13 is a diagram for explaining a procedure of the manipulation in the second embodiment.

After S1, processing in S11 is performed.

A doctor injects a substance for dissolving fiber forming the tumor part Pa into the tumor part Pa under the ultrasound guide (S11). That is, the doctor injects the substance for dissolving fiber to thereby dissolve a tumor inner portion under the ultrasound observation by the ultrasound observation device 3.

Specifically, the distal end of the needle tube 22 is introduced into the tumor inner portion under the ultrasound observation. The substance for dissolving fiber in the tumor inner portion is injected into the tumor inner portion through the needle tube 22, whereby the tumor inner portion dissolves.

The substance for dissolving fiber forming a tumor is plasmin, which is a proteolytic enzyme. Intercellular substances are decomposed by the proteolytic enzyme.

The doctor performs the injection of the plasmin into the tumor part Pa while viewing an ultrasound image as in S2 in the first embodiment.

Specifically, the plasmin, which is the substance for dissolving fiber, is poured into the syringe main body 25a in advance.

A state in which the plasmin is injected into the tumor part Pa is the same as the state shown in FIG. 6. An ultrasound image displayed on the monitor 4 at the time when the plasmin is injected into the tumor part Pa is the same as the ultrasound image shown in FIG. 7.

When the plasmin is injected into the tumor part Pa, the plasmin dissolves the fiber. The doctor injects the plasmin dividedly several times while viewing a situation of the dissolution of the fiber in the tumor part Pa by the plasmin using the ultrasound image USI displayed on the monitor 4. For example, the plasmin is injected into the tumor part Pa at an interval of several seconds.

When the plasmin is injected by a predetermined amount, for example, 0.5 to 1.0 ml, the fiber coming into contact with liquid of the plasmin pushed out from the needle tube distal end portion 22a dissolves. In the ultrasound image USI, a dissolved portion of the fiber around the needle tube distal end portion 22a changes to a black image. Therefore, the doctor can check a range of the dissolved fiber using the plasmin injected into the tumor tissue.

Subsequently, the doctor performs injection of the predetermined amount of the plasmin in order to dissolve the fiber not dissolved yet while viewing the ultrasound image.

The plasmin is additionally injected, whereby the region RA expands to be larger as indicated by dotted lines in FIGS. 6 and 7. For example, the doctor injects the plasmin dividedly several times at an interval of several seconds while viewing an ultrasound image displayed on the monitor 4.

The doctor repeats the above until the entire fiber dissolves. For example, when the doctor dissolves the entire fiber, the doctor dissolves the fiber to leave only a layer portion on an outer side surface of the fiber. The doctor can determine, by viewing the ultrasound image, whether the fiber is dissolved leaving only the layer portion on the outer side surface of the fiber.

Only the layer portion on the outer surface side of the fiber is left not to dissolve even a normal tissue. Note that a region to be dissolved may be only a desired region of the tumor part Pa rather than the entire fiber.

Therefore, the doctor injects the plasmin dividedly several times while viewing the ultrasound image until the desired region in the tumor part Pa is dissolved. An injection amount and a number of times of injection of the plasmin are adjusted according to a volume of the tumor part Pa.

When the plasmin is injected into the desired region and the desired region is dissolved, the doctor pulls out the plunger of the syringe 25 to suck the dissolved fiber and the remaining plasmin.

Subsequently, under the ultrasound guide, the doctor repeats injection of a plasmin antagonist into the tumor part Pa and suction of the plasmin antagonist from the inside of the tumor part Pa (S12).

That is, the doctor injects the plasmin antagonist, which is a solvent for suspension, into the tumor inner portion while viewing the ultrasound image. The plasmin antagonist is tranexamic acid such as Hexatron, Transamin, Recoverin, or Pletasmin, for example.

The doctor replaces the syringe 25 with a syringe into which the plasmin antagonist is poured and pushes in the plunger 25b of the syringe 25 to inject the plasmin antagonist into the region RA in which the fiber in the tumor part Pa is dissolved. Further, the doctor pulls out the plunger 25b of the syringe 25 to suck the injected plasmin antagonist. The plasmin remaining in the tumor part Pa is neutralized by the plasmin antagonist and action of the plasmin stops.

The doctor repeats the injection of the plasmin antagonist into the tumor part Pa and the suction of the plasmin antagonist from the inside of the tumor part Pa several times while viewing the ultrasound image.

As explained above, under the ultrasound observation, the doctor introduces the solvent for suspension into a tumor inner portion from the distal end of the needle tube 22 arranged in the tumor inner portion, mixes the solvent for suspension with the dissolved tumor tissue in the tumor inner portion, and changes the tumor inner portion to fluid.

Subsequently, the doctor collects the dissolved fiber together with the plasmin antagonist under the ultrasound guide (S13).

That is, after changing the tumor inner portion to the fluid, the doctor collects the tumor tissue, which mixed with the solvent for suspension softened, from the tumor inner portion using the needle tube 22. Specifically, the doctor pulls out the plunger of the syringe 25 to suck and collect the dissolved fiber and injected plasmin antagonist while viewing the ultrasound image.

When the dissolved fiber and the plasmin antagonist are sucked according to S13, since a portion where the dissolved fiber is present disappears, the volume of the tumor part Pa decreases.

A state in which the dissolved fiber and the plasmin antagonist in the tumor part Pa are sucked is the same as the state shown in FIG. 8. An ultrasound image displayed on the monitor 4 in the state in which the dissolved fiber and the plasmin antagonist in the tumor part Pa are sucked is the same as the ultrasound image shown in FIG. 9.

As shown in FIG. 8, the volume of the tumor part Pa decreases. As shown in FIG. 9, the image Pax of the tumor part Pa also decreases in volume.

Therefore, according to the present embodiment, the same effects as those in the first embodiment are generated. That is, it is possible to reduce the volume of the tumor and reduce or eliminate compression of surrounding organs, blood vessels, and nerves. It is possible to perform a surgical operation such as excision of the tumor. Further, since the tumor tissue is sucked together with the ethanol after being dissolved, there is also an effect that tumor lysis syndromes due to remaining of a cancer cell or the like in the body are suppressed. A pain due to compression of nerves based on an increase in a tumor volume is also reduced and QOL (quality of life) is also improved.

Next, modifications of the present embodiment are explained.

### (Modifications)

Note that, in the second embodiment, the configurations of the modifications 1 and 2 explained in the first embodiment can also be applied.

That is, in the present embodiment, as a modification 1, in S12, the doctor may use two needle tubes, use one needle tube for the injection of the plasmin antagonist, and use the other needle tube for the suction of the plasmin antagonist to reflux the plasmin antagonist.

Furthermore, in the present embodiment, as a modification 2, in S11, after injecting the substance for dissolving fiber into the tumor part Pa, the doctor may insert a laser probe or a heat probe through the needle tube 22 to warm the substance for dissolving fiber.

In the second embodiment, the modifications 1 and 2 generate the same effects as those in the modifications 1 and 2 in the first embodiment.

### (Modification 3)

When an amount of a tumor tissue is large and a volume of a portion where a tumor tissue is present is not reduced even if fiber of a tumor is dissolved and collected as explained above, the implementation of the first embodiment may be performed after the implementation of the present embodiment explained above. It is possible to reduce the volume of the portion where the tumor tissue is present by dissolving the tumor tissue and collecting the tumor tissue together with the ethanol after the collection of the fiber.

### Third Embodiment

In the first embodiment, the substance for dissolving a tumor tissue is injected into the tumor tissue and the dissolved tumor tissue is collected. In the second embodiment, when fibrillization of a tumor tissue advances, the substance for dissolving a tissue of the fiber is injected into the tumor tissue and the dissolved tumor tissue is collected. In a third embodiment, a fragile portion of a tumor tissue is broken up by an ultrasound vibration and the broken-up tissue is collected.

An apparatus configuration for realizing a manipulation in the present embodiment is substantially the same as the apparatus configuration in the first embodiment. The same components are denoted by the same reference signs and explanation of the components is omitted. Different components are mainly explained.

In the manipulation in the third embodiment, steps of a procedure same as steps in the first embodiment are denoted by the same reference signs and explanation of the steps is omitted.

### (Configuration)

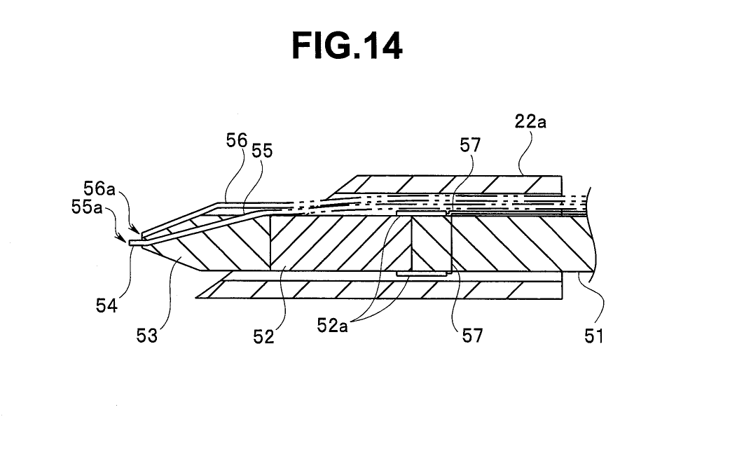

FIG. 14 is a schematic sectional view showing a configuration of an ultrasound probe inserted through a needle tube distal end portion for breaking up a tumor tissue with an ultrasound vibration.

The needle tube 22 is inserted through the instrument channel of the ultrasound endoscope 2. Further, an ultrasound probe 51 made of metal such as stainless steel is inserted through the needle tube 22. An ultrasound transducer 52 is provided to be embedded in a distal end portion of the ultrasound probe 51. A distal end portion 53 is provided on a distal end side of the ultrasound transducer 52.

The distal end portion 53 has a conical shape. A vibrating chip 54 is provided at a distal end of the conical shape. The ultrasound transducer 52 and the vibrating chip 54 configure a breaking-up tool for breaking up a tumor inner portion. Further, respective opening portions 55a and 56a of a water feeding duct 55 and a water suction duct 56 are provided at the distal end portion 53 along the ultrasound probe 51. Respective proximal end portions of the water feeding duct 55 and the water suction duct 56 are connected to a water feeding and water suction device, which is a device that feeds and sucks liquid of saline or ethanol explained below.

An electrode portion 52a is provided in the ultrasound transducer 52. A signal line 57 connected to an ultrasound control device explained below is connected to the electrode portion 52a.

When the ultrasound transducer 52 vibrates at a predetermined ultrasound frequency, the vibrating chip 54 of the distal end portion 53 ultrasonically vibrates. A frequency of the ultrasound transducer 52 is, for example, about 1/100 to 1/1000 of a frequency for an ultrasound image. If the frequency for an ultrasound image is 7.5 MHz, the frequency of the ultrasound transducer 52 is 23.5 to 47 KHz.

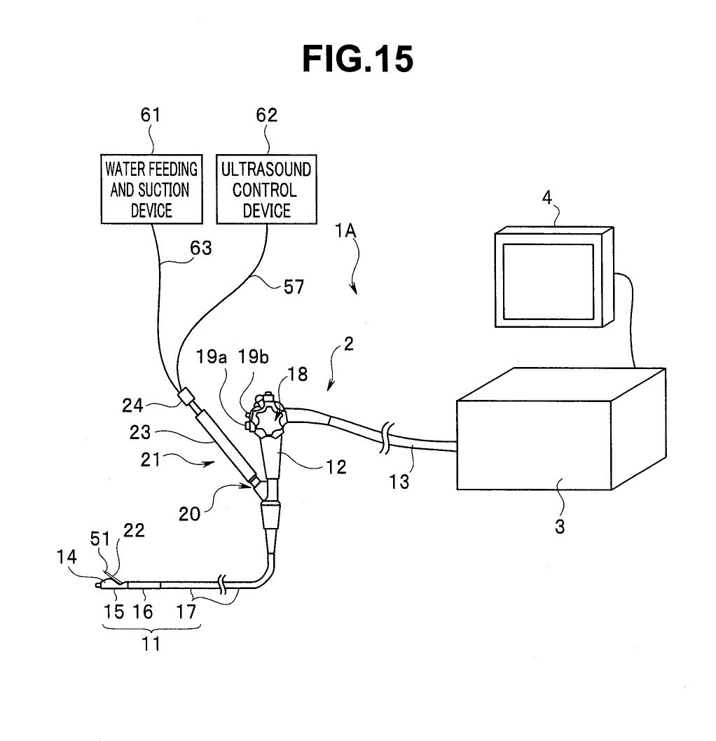

FIG. 15 is a configuration diagram showing a configuration of an ultrasound endoscope apparatus 1A according to the present embodiment. The ultrasound endoscope apparatus 1A includes a water feeding and water suction device 61 connected to a duct 63 including the water feeding duct 55 and the water suction duct 56 provided in the ultrasound probe 51 shown in FIG. 14 and an ultrasound control device 62 to which the signal line 57 extending from the ultrasound transducer 52 of the ultrasound probe 51 is connected.

By operating a switch (not shown in the figure) provided in the water feeding and water suction device 61, a doctor can feed liquid (saline or ethanol as explained below) via the water feeding duct 55, eject the liquid from a distal end of the ultrasound probe 51, and suck the liquid from the distal end of the ultrasound probe 51 via the water suction duct 56.

By operating a switch (not shown in the figure) provided in the ultrasound control device 62, the doctor can drive the ultrasound transducer 52.

### (Manipulation)

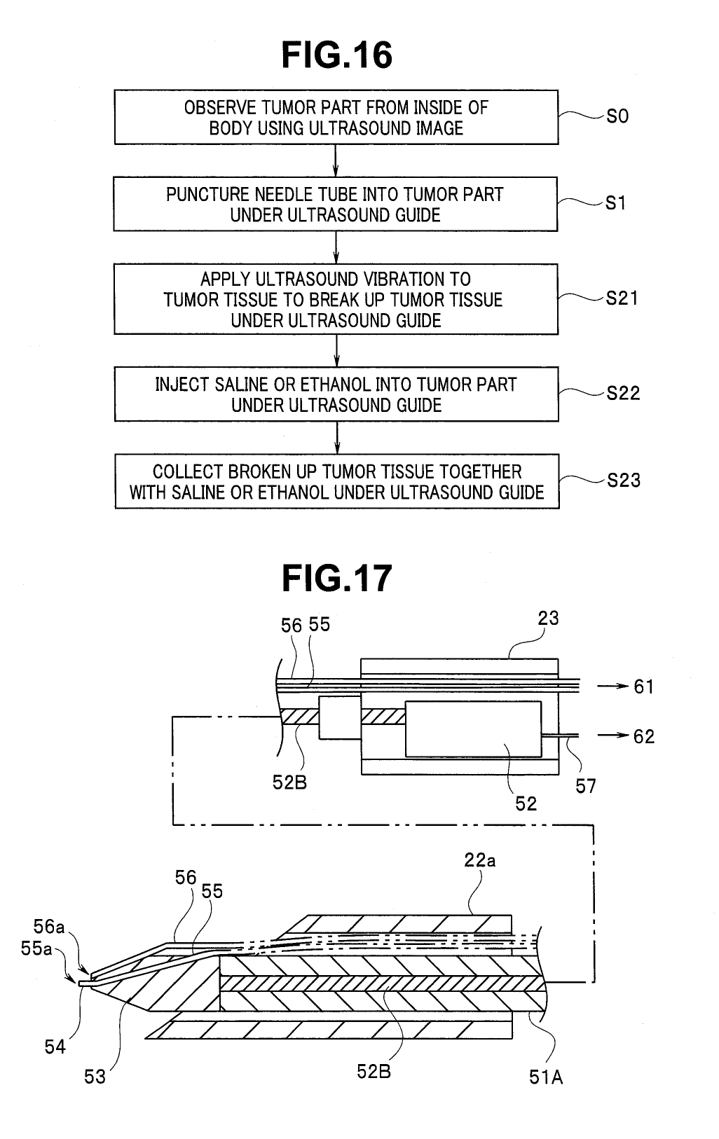

FIG. 16 is a diagram for explaining a procedure of a manipulation in the third embodiment.

After S1, processing in S21 is performed. Since the distal end portion of the ultrasound probe 51 is inserted through the needle tube 22, the doctor applies an ultrasound vibration to a tumor tissue to break up the tumor tissue under the ultrasound guide (S21). That is, the doctor breaks up a tumor inner portion by applying the ultrasound vibration to the tumor tissue under the ultrasound observation by the ultrasound observation device 3. Specifically, the tumor inner portion is broken up by the ultrasound transducer 52 and the vibrating chip 54 configuring the breaking-up tool for breaking up the tumor inner portion.

The distal end portion of the ultrasound probe 51 is vibrated by driving the ultrasound transducer 52, which is an ultrasound probe separate from the ultrasound probe 14 for generating an ultrasound image. Breaking up of the tumor tissue is performed by the vibration. In particular, a fragile portion of the tumor tissue is easily broken up by the ultrasound vibration.

For example, the doctor can check a situation of the break-up by the ultrasound vibration viewing an ultrasound image displayed on the monitor 4.

For example, the doctor can determine, by viewing the ultrasound image USI displayed on the monitor 4, whether the entire tumor tissue is broken up. When the tumor tissue is broken up, air or the like enters among tissues, gaps are formed, and an image of a portion of the tumor tissue is brightened. Therefore, the doctor can determine, by viewing the ultrasound image, whether the tumor tissue is broken up. As explained above, the doctor repeatedly applies the ultrasound vibration to the tumor tissue viewing the ultrasound image until the entire tumor tissue or a predetermined region is broken up.

Subsequently, the doctor injects saline or ethanol, which is a solvent for suspension, into the tumor part Pa under the ultrasound guide (S22).

When the break-up of the tumor tissue ends in S21, the doctor starts the water feeding and water suction device 61 and injects the saline or the ethanol into the tumor part Pa.

As explained above, the doctor introduces the solvent for suspension into the tumor inner portion from the distal end of the needle tube 22 arranged in the tumor inner portion, mixes the solvent for suspension with the broken-up tumor tissue in the tumor inner portion, and changes the tumor inner portion to fluid under the ultrasound observation.

The doctor collects the broken-up tumor tissue together with the saline or the ethanol under the ultrasound guide (S23).

When the saline or the ethanol is injected in S22, fine fragments of the broken-up tumor tissue float in the saline or the ethanol. Therefore, a volume of a portion where the tumor tissue is present is reduced by sucking the broken-up tumor tissue together with the saline or the ethanol. That is, after changing the tumor inner portion to the fluid, the doctor collects the tumor tissue, which is mixed with the solvent for suspension and softened, from the tumor inner portion using the water suction duct 56.

Note that, when the volume of the tumor does not decrease to a desired volume even if the broken-up tumor tissue is sucked, the doctor may perform the procedure of (S21) to (S23) explained above again and repeat the procedure of (S21) to (S23) until the volume of the tumor does not decrease to the desired volume.

Therefore, according to the present embodiment, the same effects as those in the first embodiment are generated. That is, it is possible to reduce the volume of the tumor and reduce or eliminate compression of surrounding organs, blood vessels, and nerves. It is possible to perform a surgical operation such as excision of the tumor. Further, since the tumor tissue is sucked together with the ethanol after being dissolved, there is also an effect that tumor lysis syndromes due to remaining of a cancer cell or the like in the body are suppressed. A pain due to compression of nerves based on an increase in a tumor volume is also reduced and QOL (quality of life) is also improved.

Note that, in the example explained above, the saline or the like is injected into the tumor part and the injected saline or the like is sucked using the water feeding and water suction device 61. However, as shown in FIG. 1, the doctor may perform the injection and the suction of the saline or the like using a syringe. It is possible to inject the saline or the ethanol into the tumor part Pa by pouring the saline or the ethanol into the syringe main body 25a and pushing in the plunger 25b. It is possible to suck the saline or the ethanol from the inside of the tumor part Pa by pulling out the plunger 25b.

Next, modifications of the present embodiment are explained.

### (Modification 4)

Note that the ultrasound probe inserted through the needle tube distal end portion 22a may have a configuration shown in FIG. 17. FIG. 17 is a schematic sectional view showing a configuration of a modification of an ultrasound probe inserted through a needle tube distal end portion for breaking up a tumor tissue with an ultrasound vibration.

A vibration transmitting member 52B is included in an ultrasound probe 51A according to this modification. A distal end of the vibration transmitting member 52B is connected to the distal end portion 53. A proximal end of the vibration transmitting member 52B is connected to an ultrasound transducer 52A provided in the main body portion 23. The ultrasound transducer 52A is connected to the ultrasound control device 62 via a signal line 57A. The other components are the same as the components in the third embodiment explained above.

In the case of this modification, the ultrasound transducer 52A is provided in the main body portion 23 of the puncture device 21. A vibration of the ultrasound transducer 52A is transmitted to the distal end portion 53 of the ultrasound probe 51A by the vibration transmitting member 52B. The same effects as those in the present embodiment are generated by such a configuration.

### Fourth Embodiment

In the first to third embodiments, the tumor tissue is dissolved or broken up by the ultrasound vibration and the dissolved or broken up tumor tissue is collected. However, in a fourth embodiment, a tumor tissue is resected by a blade and the resected tumor tissue is collected.

An apparatus configuration for realizing a manipulation in the present embodiment is substantially the same as the apparatus configuration in the first embodiment. The same components are denoted by the same reference signs and explanation of the components is omitted. Different components are mainly explained.

In the manipulation in the fourth embodiment, steps of a procedure same as in the first embodiment are denoted by the same reference signs and explanation of the steps is omitted.

### (Configuration)

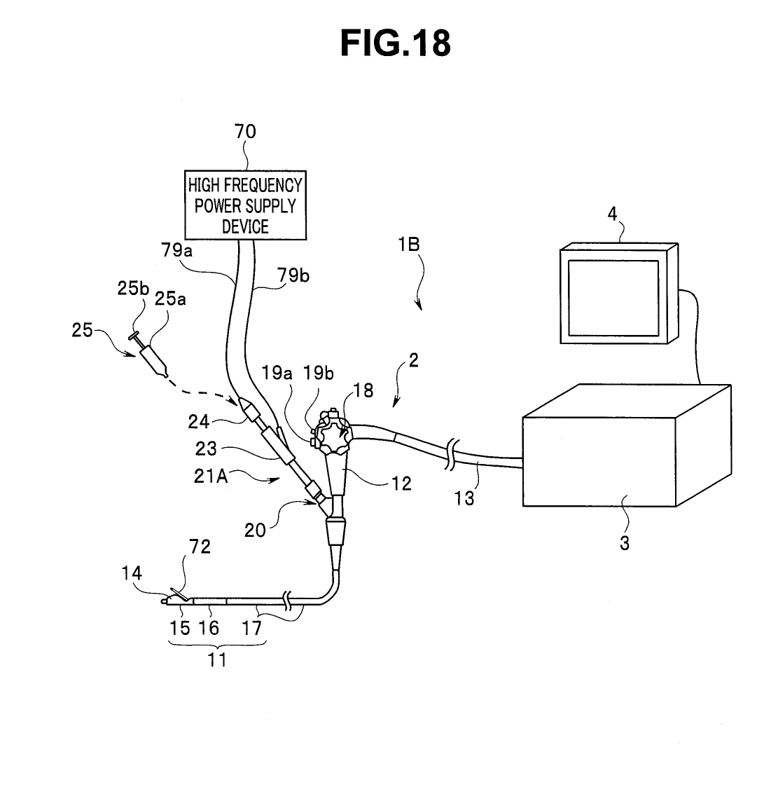

FIG. 18 is a configuration diagram showing a configuration of an ultrasound endoscope apparatus 1B according to the present embodiment. A high frequency power supply device 70 is connected to the puncture device 21A. The high frequency power supply device 70 can supply a high frequency current to the needle tube 71 and a blade 72 as explained below. The blade 72 is a resecting tool for resecting a tumor inner portion. The puncture device 21A is connected to the high frequency power supply device 70 via two cables 79a and 79b. Note that a counter electrode plate (not shown in the figure), which comes into contact with a patient, is disposed for time when a high frequency current is fed to the puncture device 21A. The supply of the high frequency current to each of the blade 72 and the needle tube 71 can be performed according to operation of a switch (not shown in the figure) provided in the high frequency power supply device 70 by a doctor.

Further, the syringe 25 can be attached to the puncture device 21A.

The puncture device 21A includes an elongated puncture tool insertion portion 79A, through which the needle tube 71 and the blade 72 are inserted, and a puncture tool operation portion 79B provided on a proximal end side of the puncture tool insertion portion 79A and for performing projecting operation for the needle tube 71 and the like. The puncture device 21A is configured such that the puncture tool insertion portion 79A can be inserted from the treatment instrument insert-through port 20 of the endoscope operation portion 12 and a distal end portion of the puncture tool insertion portion 79A can be projected from the treatment instrument opening of the distal end rigid portion 15 through the instrument channel in the endoscope insertion portion 11.

First, a configuration of the puncture tool insertion portion 79A is explained. The puncture tool insertion portion 79A includes the sheath 31 and the needle tube 71 and the blade 72 inserted through the sheath 31.

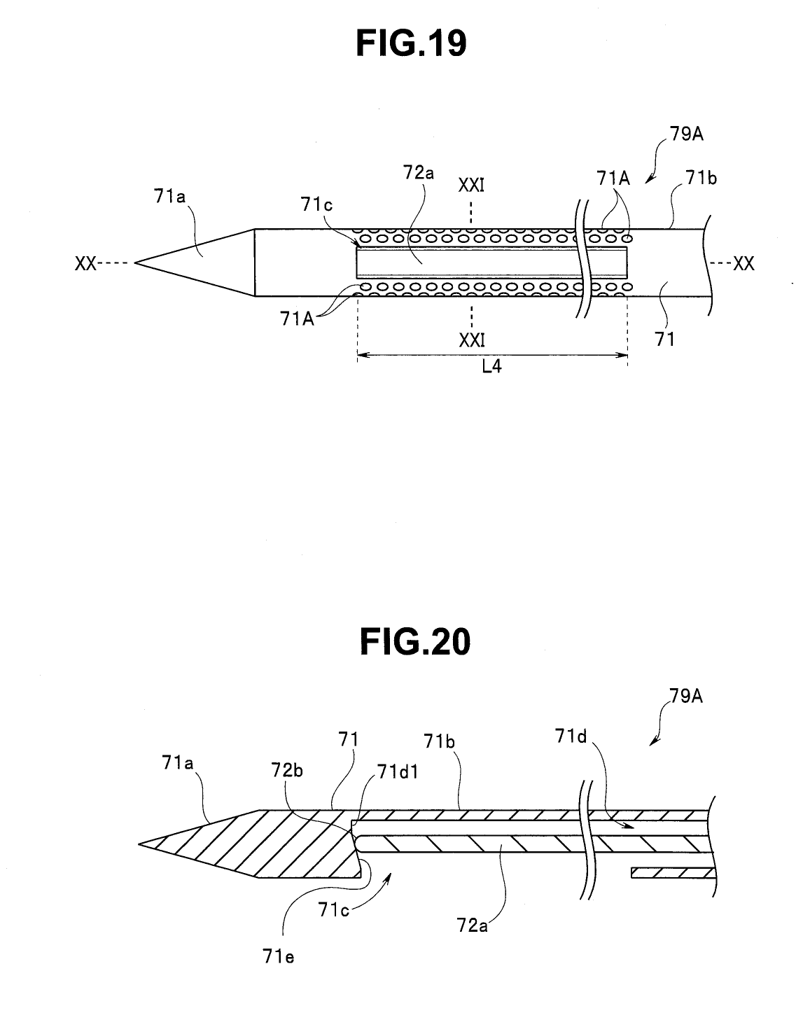

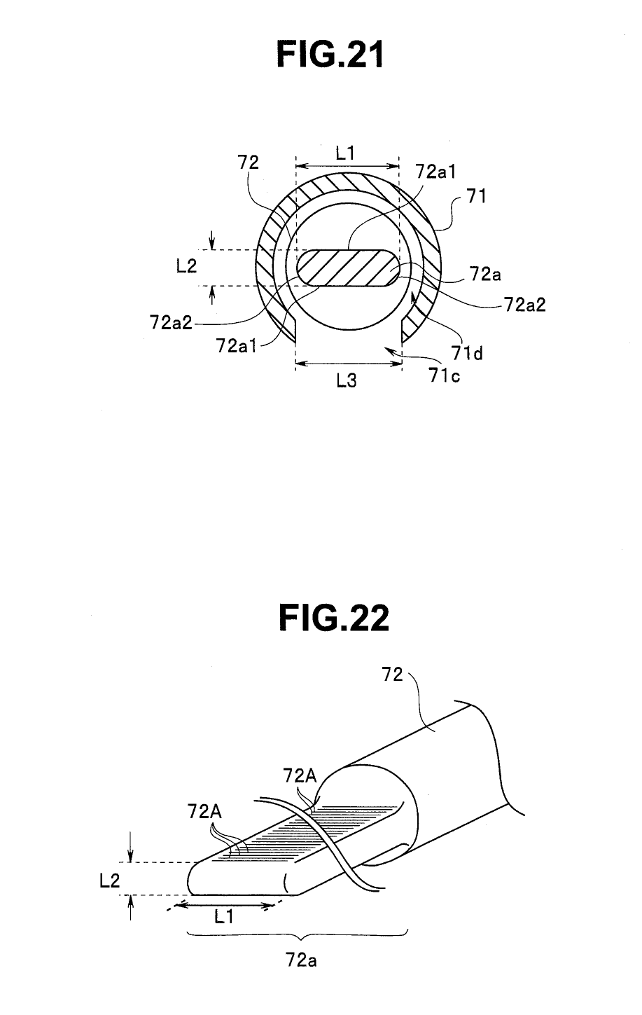

FIG. 19 is a plan view of the distal end portion of the puncture tool insertion portion 79A. FIG. 20 is a sectional view of the distal end portion of the puncture tool insertion portion 79A taken along line XX-XX in FIG. 19. FIG. 21 is a sectional view of the distal end portion of the puncture tool insertion portion 79A taken along line XXI-XXI in FIG. 19. FIG. 22 is a perspective view of a distal end portion of the blade 72.

The puncture tool insertion portion 79A includes the sheath 31, the needle tube 71 inserted through the sheath 31, and the blade 72 inserted through the needle tube 71.

As shown in FIGS. 19 and 20, the needle tube 71 is a tubular member of stainless steel, nickel titanium, a cobalt chrome alloy, or the like, a hole at a distal end portion of which is closed by welding or the like. That is, a tubular portion including a channel on an inner portion is configured in the needle tube 71. The hollow and elongated needle tube 71 includes a needle tube distal end portion 71b including a sharp conical shape portion 71a at a distal end. The needle tube distal end portion 71b includes an elongated opening portion 71c formed along an axial direction. The opening portion 71c is provided in a vicinity of the conical shape portion 71a: That is, the opening portion 71c is provided on a proximal end side of a distal end portion of the needle tube distal end portion 71b and communicates with the channel in the needle tube 71. The conical shape portion 71a is arranged at the distal end portion of the needle tube 71, which is the tubular portion, and configures a puncture portion to be punctured into a subject. A diameter of the needle tube 71 is, for example, 22 G to 19 G (gauge). Length in an axial direction of the opening portion 71c is, for example, 5 to 15 mm. That is, a distal end of the needle tube 71 has a so-called pencil shape.

The blade 72, which is an elongated shaft member having a circular section, is inserted through the internal space 71d of a hollow needle tube distal end portion 71b. A diameter of a shaft portion of the blade 72 is, for example, 0.5 to 1 mm. The blade 72 is also made of metal such as stainless steel, nickel titanium, or a cobalt chrome alloy.

Machining for oxidizing a surface to increase contact resistance and reduce electric conductivity with electric discharge machining or machining for forming an insulating film by surface coating using polyimide or the like is applied to a channel inner side surface of the needle tube distal end portion 71b.

Alternatively, the machining for increasing the contact resistance or the machining for forming the insulating film may be applied to a surface of the blade 72 in a portion other than a portion in contact with a distal end side inner wall portion 71e at the blade distal end portion 72a and a portion projecting from the opening portion 71c of the blade distal end portion 72a. Note that, in this case, the machining for increasing the contact resistance or the machining for forming the insulating film may be applied to a channel inner side surface of the needle tube 71 as well.

As explained above, the machining for reducing electric conductivity between the blade 72 and an inner wall of the needle tube distal end portion 71b or the needle tube 71 having the puncture portion or the machining for performing electric insulation between the blade 72 and the inner wall of the needle tube distal end portion 71b or the needle tube 71 is applied to at least one of the blade 72 and the needle tube 71.

As shown in FIG. 21, a sectional shape in a direction orthogonal to an axial direction of the blade distal end portion 72a of the blade 72 includes two plane portions 72a1 and semicircular portions 72a2 at both ends of the two plane portions 72a1. That is, the blade distal end portion 72a includes a flat portion having a flat section and a thin extending tabular shape. Width L1 of the tabular shape is, for example, 0.3 to 0.9 mm. Thickness L2 of the tabular shape is, for example, 0.1 to 0.2 mm.

As shown in FIG. 20, a curved surface portion 72b semicircular in a sectional shape extending along the axial direction of the blade 72 is provided at a distal end of the blade distal end portion 72a. Further, ultrasound reflection machining for reflecting ultrasound is applied to the plane portions 72a1. As shown in FIG. 22, a large number of fine grooves 72A are formed as ultrasound reflection machined portions in the two plane portions 72a1.

Note that, as explained below, the large number of grooves 72A of the ultrasound reflection machined portions may have a shape formed in a range in which the blade distal end portion 72a bends in an arcuate shape and formed along a direction orthogonal to the axial direction of the blade distal end portion 72a and contributing to bending of the blade distal end portion 72a.

The width L1 of the blade distal end portion 72a is smaller than width L3 of the opening portion 71c of the needle tube 71. As explained below, the blade distal end portion 72a and the opening portion 71c are configured such that, when the blade distal end portion 72a bends, a bending portion of the blade distal end portion 72a can project from an opening portion 71c. The bending portion formed when the blade distal end portion 72a projects from the opening portion 71c configures a cutting portion.

As explained above, the blade 72 can be inserted through the channel of the needle tube 71. The blade 72 is configured such that, when the blade 72 is inserted through the channel, the bending portion functioning as the cutting portion can project from the opening portion 71c.

As shown in FIG. 19, the ultrasound reflection machining is applied to the needle tube distal end portion 71b over a predetermined range from a distal end portion to a proximal end portion of the opening portion 71c. Specifically, dimple machining of a plurality of dimples 71A is applied as ultrasound reflection machined portions over a range L4 in which the opening portion 71c is present on an outer side surface of the needle tube distal end portion 71b.

Note that, although the ultrasound reflection machined portions of the plurality of dimples 71A are formed over the range L4 in which the opening portion 71c is present, the ultrasound reflection machined portions may be provided only in two places of the distal end portion of the opening portion 71c and the proximal end portion of the opening portion 71c such that the dimple machining is absent between the distal end portion and the proximal end portion of the opening portion 71c.

The distal end side inner wall portion 71e of the needle tube distal end portion 71b forming the internal space 71d has a slope inclined at a predetermined angle with respect to a surface orthogonal to an axial direction of the needle tube 71. A wall surface of the distal end side inner wall portion 71e is formed as a slope extending closer to a distal end direction of the needle tube 71 from the distal end portion 71d1 of the internal space 71d toward the opening portion 71c. In other words, the wall surface of the distal end side inner wall portion 71e is formed as a slope extending closer to the distal end portion of the needle tube 71 from the opening portion 71c toward depth of the internal space 71d. Therefore, the needle tube distal end portion 71b has an inclined surface for preventing the blade distal end portion 72a from projecting from the opening portion 71c when the bending portion of the blade distal end portion 72a is formed.

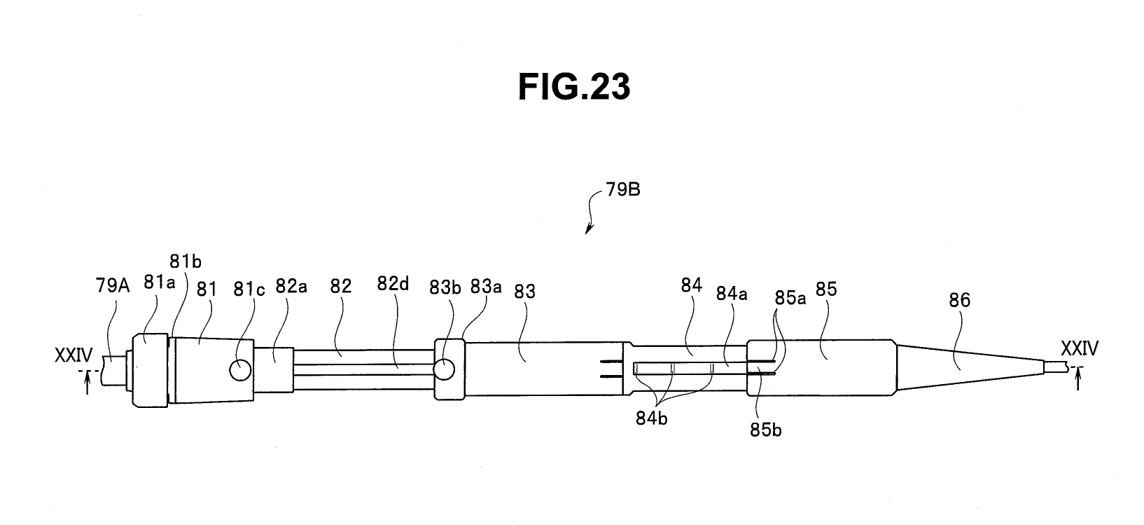

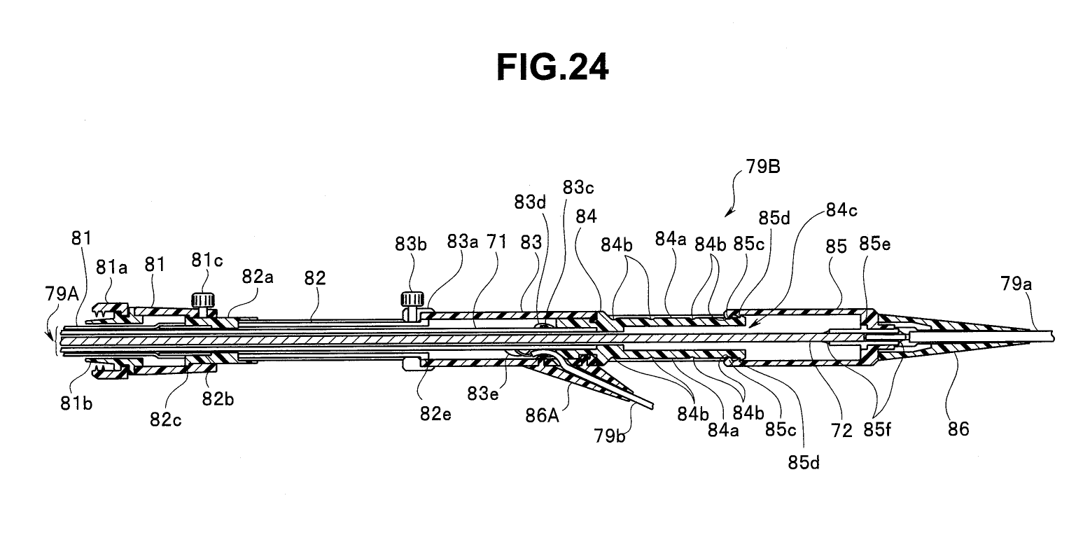

Next, a configuration of the puncture tool operation portion 79B connected to a proximal end portion of the puncture tool insertion portion 79A is explained. FIG. 23 is an external view of the puncture tool operation portion 79B. FIG. 24 is a sectional view of the puncture tool operation portion 79B taken along line XXIV-XXIV of FIG. 23.

The puncture tool operation portion 79B is attached and fixed to the treatment instrument insert-through port 20 of the ultrasound endoscope 2. A connecting portion 81 for attachment to the treatment instrument insert-through port 20 is provided at a distal end portion of the puncture tool operation portion 79B. Further, the puncture tool operation portion 79B includes a main body 82, a needle tube slider 83, a needle tube turning operation portion 84, a blade slider 85, and a bend preventing portion 86 for protecting a cable 79a in order toward a proximal end side.

The connecting portion 81 includes a connection ring 81a and a distal end connecting member 81b on a distal end side. The connecting portion 81 is attached to the treatment instrument insert-through port 20 of the endoscope operation portion 12 to internally insert the distal end connecting member 81b into the treatment instrument insert-through port 20 and the connection ring 81a is turned in a predetermined direction, whereby the puncture tool operation portion 79B can be fixed to the endoscope operation portion 12. A sheath fixing knob 81c is provided on a proximal end side of the connecting portion 81.

A connecting member 82a is provided on a distal end side of the main body 82. The sheath 31 of the puncture tool insertion portion 79A is externally inserted over and fixed to a connection pipe 82b provided at a distal end of the connecting member 82a. The connecting member 82a is loosely fit in the connecting portion 81 having a cylindrical shape. The sheath 31 can be fixed to the connecting portion 81 in a desired position by turning the sheath fixing knob 81c in a predetermined direction. A stopper 82c that comes into contact with an inner surrounding side convex portion of the cylindrical connecting portion 81 to prevent the connecting member 82a from coming off the connecting portion 81 is provided on a distal end side of the connecting member 82a.

A main body groove portion 82d is formed along an axial direction of the main body 82 on an outer surface of the main body 82. A connecting member 83a is provided on a distal end side of the needle tube slider 83. A stopper 82e that comes into contact with an inner surrounding side convex portion of a cylindrical connecting member 83a to prevent the main body 82 from coming off the needle tube slider 83 is provided on a proximal end side of the main body 82.

A needle tube fixing knob 83b is provided in the connecting member 83a. On a proximal end side, the main body 82 is loosely fit in the needle tube slider 83 having a cylindrical shape. The needle tube slider 83 can be fixed to the main body 82 in a desired position by turning the needle tube fixing knob 83b in a predetermined direction.

In a proximal end portion of the needle tube slider 83, the needle tube turning operation portion 84 having a cylindrical shape is provided to be engaged with the needle tube slider 83 to be capable of turning around an axis of the needle tube turning operation portion 84. Two blade adjusting grooves 84a are provided along an axial direction of the needle tube turning operation portion 84 on an outer surface of the needle tube turning operation portion 84. Further, a plurality of (four here) concave portions 84b are provided at a predetermined interval along an axial direction in the respective blade adjusting grooves 84a.

The needle tube 71 is inserted through the sheath 31 of the puncture tool insertion portion 79A. The blade 72 is inserted through the needle tube 71. A proximal end of the needle tube 71 is fixed to a distal end portion of the needle tube turning operation portion 84. Therefore, when the needle tube turning operation portion 84 is turned around an axis, the needle tube 71 also turns around an axis.

The blade slider 85 having a cylindrical shape is externally inserted over and provided on a proximal end side of the needle tube turning operation portion 84. On a distal end side of the blade slider 85, two engaging portions 85b held between two grooves 85a formed along an axial direction are provided. Two convex portions 85c projecting to an inner side and stoppers 85d projecting to the inner side are provided in the engaging portion 85b. The two convex portions 85c engage with the two blade adjusting grooves 84a. The respective convex portions 85c are formed in the blade slider 85 to press outer side surfaces of the blade adjusting grooves 84a and to be movable along the axial direction of the needle tube turning operation portion 84.

A cylindrical blade fixing cap 85e is fixed and provided on the proximal end side of the blade slider 85. A proximal end portion of the blade 72 is inserted from a distal end side of the blade fixing cap 85e. A signal line of the cable 79a is inserted from a rear end side of the blade fixing cap 85e. The blade 72 and the cable 79a are soldered and fixed by solder 85f. Therefore, the blade 72 is fixed to the blade slider 85 and electrically connected to the cable 79a by the blade fixing cap 85e. That is, the cable 79a connected to the blade 72 configures a power supply connecting portion for electrically connecting the blade distal end portion 72a, which is the cutting portion of the blade 72, and the high frequency power supply device 70, which is a power supply.

Therefore, when the doctor moves the blade slider 85 along the axial direction of the needle tube turning operation portion 84, the blade slider 85 is lightly fixed to the needle tube turning operation portion 84 in a position where the convex portions 85c engage with the concave portions 84b. However, the blade slider 85 can be moved along the axial direction of the needle tube turning operation portion 84 with a stronger force. The stopper 85d is a stopper that comes into contact with an outer surrounding side convex portion of the needle tube turning operation portion 84 to prevent the blade slider 85 from coming off the needle tube turning operation portion 84.

Further, when the blade slider 85 is moved to a proximal end side to separate the engaging portion 85b from a surface of the needle tube turning operation portion 84 and cause the stopper 85d to climb over the outer surrounding side convex portion of the needle tube turning operation portion 84, the blade slider 85 can be detached from the needle tube turning operation portion 84 and the blade 72 can be pulled out from the needle tube 71.

When the blade slider 85 is pulled out, the syringe 25 can be attached to an opening portion 84c at a proximal end portion of the needle tube turning operation portion 84. That is, the opening portion 84c at the proximal end portion of the needle tube turning operation portion 84 configures a suction device connecting portion for connecting the syringe 25, which is a suction device arranged on a proximal end side of the channel of the needle tube 71.

The cable 79b extends from a side surface portion of the needle tube slider 83. A bend preventing portion 86A is provided in the needle tube slider 83 to protect surroundings of the cable 79b. A distal end portion of the cable 79b is pressed against the needle tube 71 by a binder 83c, which is a fixing member. The distal end portion of the cable 79b is fixed to the needle tube 71 by applying an adhesive 83d around the binder 83c. The signal line of the cable 79b is soldered to the needle tube 71 by solder 83e on an inner portion of the needle tube slider 83. A connector for connecting the cable 79b to the high frequency power supply device 70 is provided at a proximal end portion of the cable 79b. Therefore, the cable 79b connected to the needle tube distal end portion 71b including the puncture portion configures a power supply connecting portion for electrically connecting the needle tube distal end portion 71b including the conical shape portion 71a, which is the puncture portion, and the high frequency power supply device 70, which is the power supply.

Since the puncture tool operation portion 79B has the configuration explained above, the doctor can project and retract, from the treatment instrument opening of the distal end rigid portion 15 of the endoscope insertion portion 11, each of the sheath 31, the needle tube 71, and the blade 72 of the puncture tool insertion portion 79A inserted through the instrument channel of the ultrasound endoscope 2 by operating the puncture tool operation portion 79B.

FIG. 23 and FIG. 24 show a state in which the sheath 31, the needle tube 71, and the blade 72 are drawn into the most proximal end side of the puncture tool insertion portion 79A. When the puncture device 21A is attached to the treatment instrument insert-through port 20, in the state shown in FIG. 23 and FIG. 24, the sheath 31, the needle tube 71, and the blade 72 do not project from the treatment instrument opening of the distal end rigid portion 15 of the endoscope insertion portion 11.

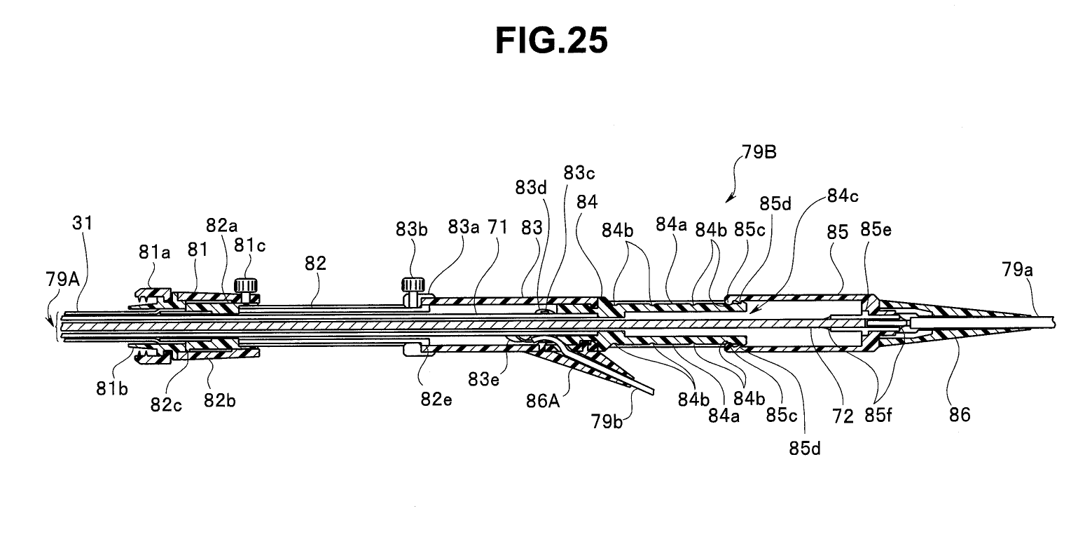

The doctor can change the puncture tool operation portion 79B from the state shown in FIG. 23 and FIG. 24 to a state shown in FIG. 25 by moving the main body 82 to the distal end side with respect to the connecting portion 81. FIG. 25 is a sectional view of the puncture tool operation portion 79B in a state in which the sheath 31 projects most from the treatment instrument opening of the distal end rigid portion 15.

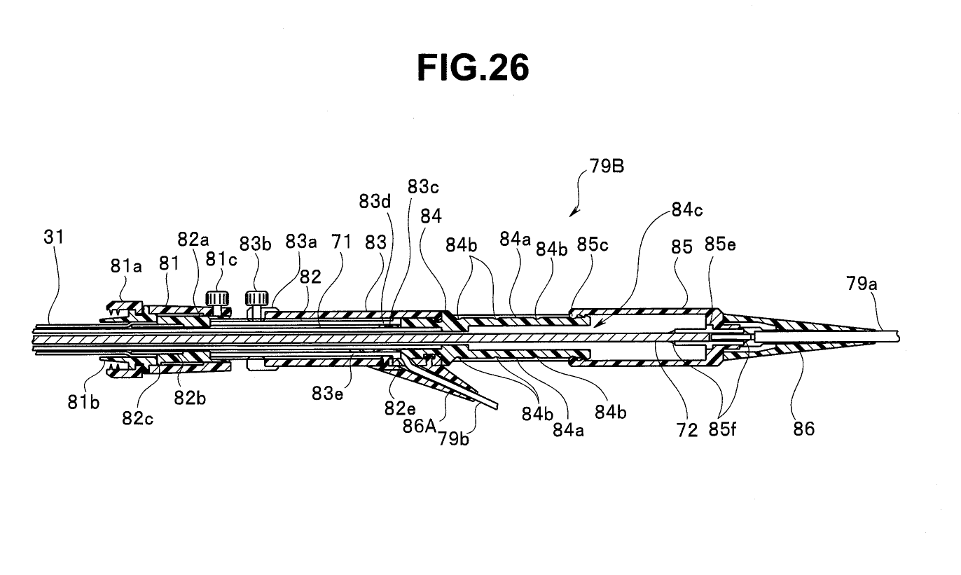

The doctor can change the puncture tool operation portion 79B from the state shown in FIG. 25 to a state shown in FIG. 26 by moving the needle tube slider 83 to the distal end side with respect to the main body 82. FIG. 26 is a sectional view of the puncture tool operation portion 79B in a state in which the sheath 31 and the needle tube 71 project most from the treatment instrument opening of the distal end rigid portion 15.

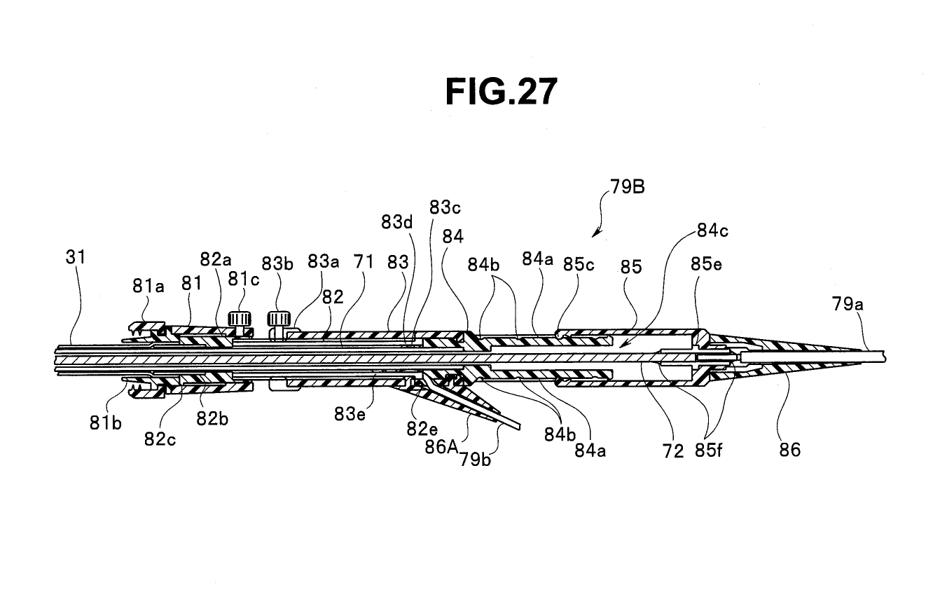

The doctor can change the puncture tool operation portion 79B from the state shown in FIG. 26 to a state shown in FIG. 27 by moving the blade slider 85 to the distal end side with respect to the needle tube turning operation portion 84. FIG. 27 is a sectional view of the puncture tool operation portion 79B in a state in which the sheath 31 and the needle tube 71 project most from the treatment instrument opening of the distal end rigid portion 15 and the blade 72 is projected by one stage from the treatment instrument opening of the distal end rigid portion 15.

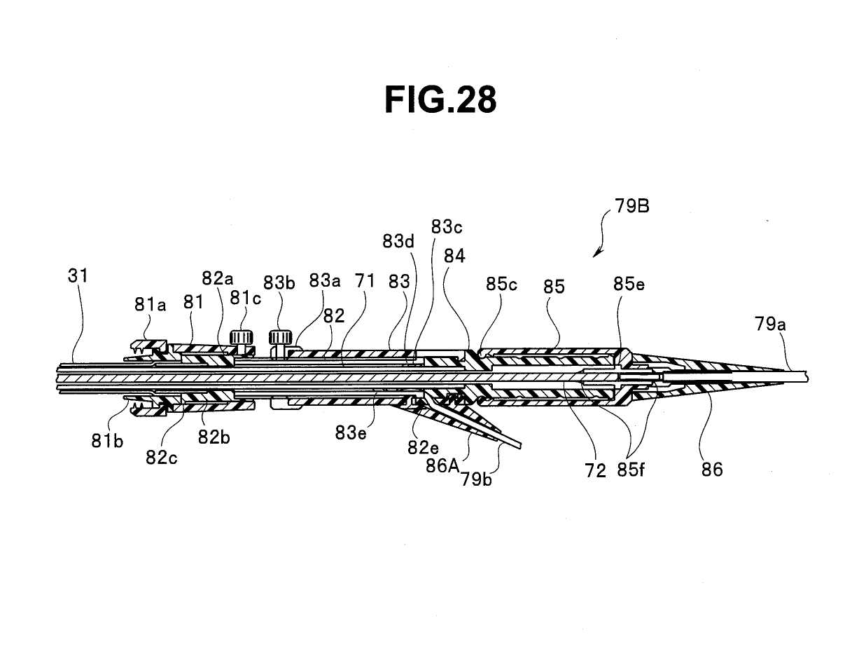

The doctor can change the puncture tool operation portion 79B from the state shown in FIG. 27 to a state shown in FIG. 28 by further moving the blade slider 85 to the distal end side with respect to the needle tube turning operation portion 84. FIG. 28 is a sectional view of the puncture tool operation portion 79B in a state in which the sheath 31 and the needle tube 71 project most from the treatment instrument opening of the distal end rigid portion 15 and the blade 72 is projected most from the treatment instrument opening of the distal end rigid portion 15.

When the puncture tool operation portion 79B is in the state shown in FIG. 27, the blade distal end portion 72a bends and the bending portion of the blade distal end portion 72a projects from the opening portion 71c. When the puncture tool operation portion 79B is in the state shown in FIG. 28, the blade distal end portion 72a further bends and the bending portion, which is the cutting portion, further projects from the opening portion 71c.

Therefore, the doctor can project each of the sheath 31, the needle tube 71, and the blade 72 at the distal end portion of the puncture tool insertion portion 79A by a desired amount from the treatment instrument opening of the distal end rigid portion 15 and retract each of the sheath 31, the needle tube 71, and the blade 72 into the treatment instrument opening of the distal end rigid portion 15 by operating the respective portions of the puncture tool operation portion 79B.

When the blade 72 is pushed out to the distal end side, the curved surface portion 72b semicircular in a sectional shape comes into contact with the distal end side inner wall portion 71e. When the blade 72 is further pushed out to the distal end side, the curved surface portion 72b moves along the slope of the distal end side inner wall portion 71e and comes into contact with the distal end portion 71 dl of the internal space 71d. Therefore, the blade distal end portion 72a bends to project from the opening portion 71c.

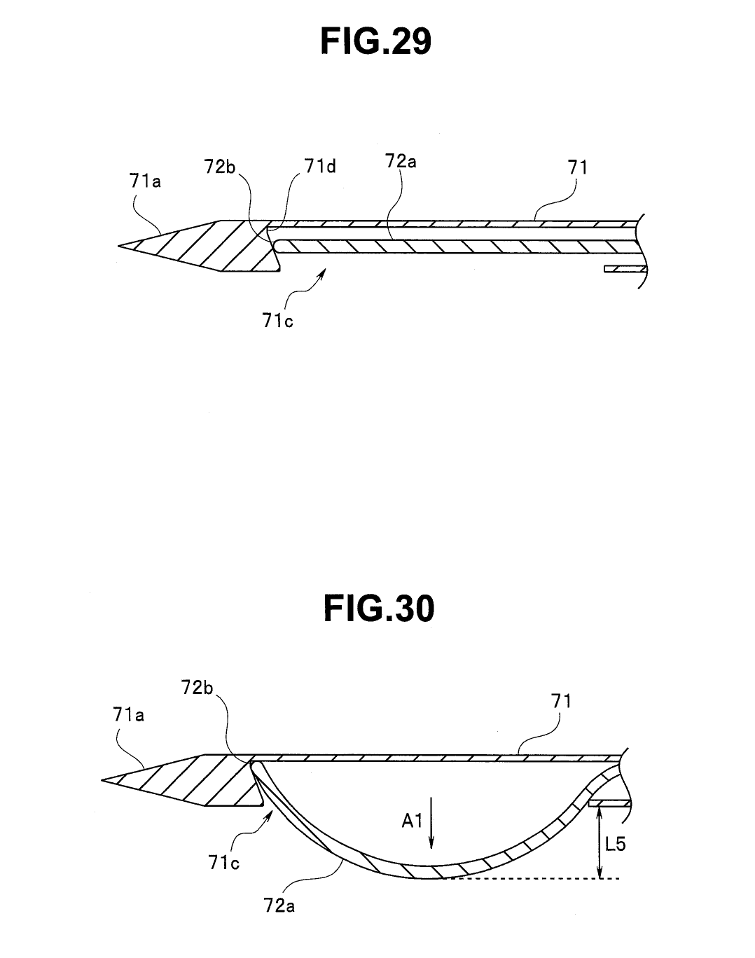

FIG. 29 is a sectional view showing a state in which the blade distal end portion 72a comes into contact with the distal end portion 71d1 of the internal space 71d but the blade distal end portion 72a is not bent. FIG. 30 is a sectional view showing a state in which the blade distal end portion 72a comes into contact with the distal end portion 71d1 of the internal space 71d and the blade 72 is further pushed out to the distal end side and the blade distal end portion 72a is bent.

A projection amount L5 of the blade distal end portion 72a changes according to a movement amount of the blade slider 85 to the distal end side with respect to the needle tube turning operation portion 84. Respective positions of the concave portions 84b correspond to projection amounts of the blade distal end portion 72a. Therefore, the doctor can change the projection amount L5 of the blade distal end portion 72a by moving the blade slider 85 along the axial direction of the needle tube turning operation portion 84 and changing the positions of the concave portions 84b of the needle tube turning operation portion 84 with which the respective convex portions 85c of the blade slider 85 engage. As the doctor moves the blade slider 85 further to the distal end side with respect to the needle tube turning operation portion 84, the blade distal end portion 72a projects further in a direction indicated by an arrow A1 and the projection amount L5 of the blade distal end portion 72a from the opening portion 71c increases.

### (Manipulation)

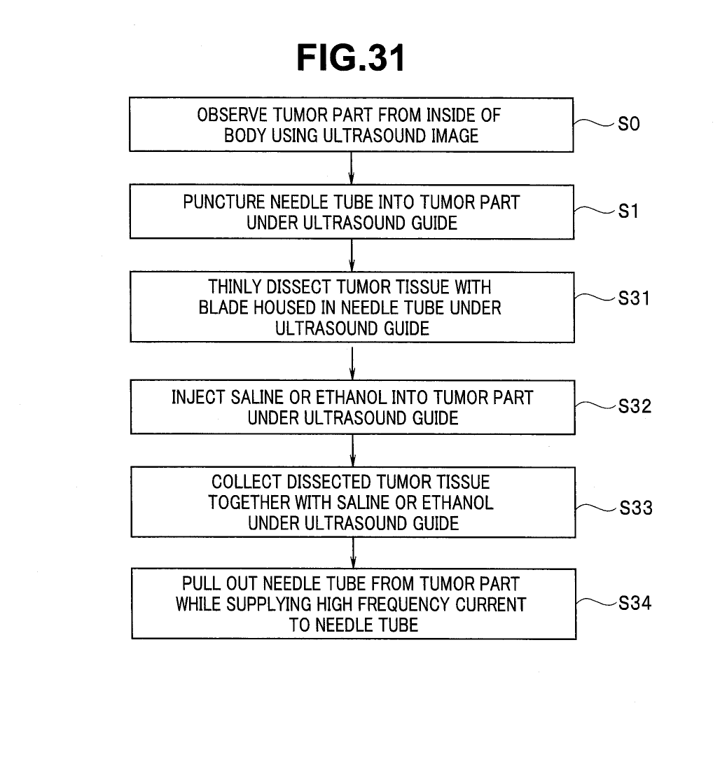

FIG. 31 is a diagram for explaining a procedure of a manipulation in the fourth embodiment.

After S1, processing in S31 is performed. Subsequently, the doctor thinly resects a tumor tissue with the blade 72 housed in the needle tube 71 under the ultrasound guide (S31). That is, the doctor resects a tumor inner portion by shaving the tumor tissue with the blade 72, which is the resecting tool for resecting the tumor inner portion under the ultrasound observation by the ultrasound observation device 3.

For example, the doctor can resect the tumor tissue by projecting the blade distal end portion 72a while viewing an ultrasound image displayed on the monitor 4 and checking a projecting state of the bending portion of the blade distal end portion 72a and by turning the needle tube 71 little by little around the axis while supplying a high frequency current to the blade 72.

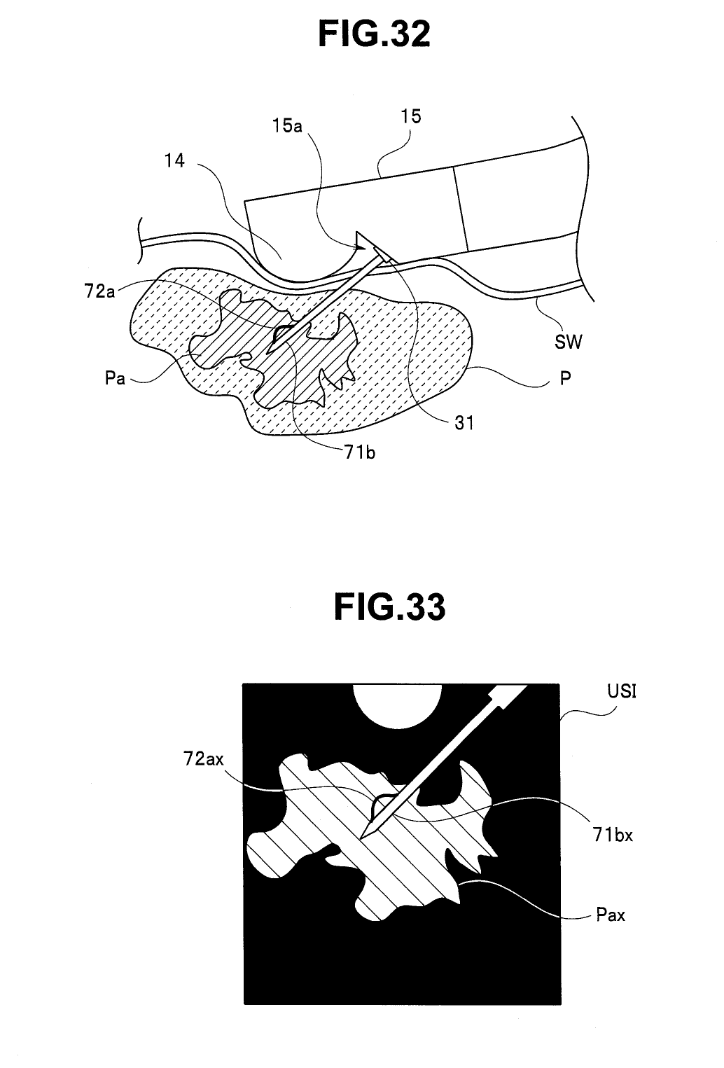

Specifically, the doctor can project the blade distal end portion 72a from the opening portion 71c by moving the blade slider 85 to the distal end side. FIG. 32 is a diagram for explaining a projecting state of the blade distal end portion 72a. As shown in FIG. 32, the needle tube distal end portion 71b pierces through the stomach wall SW and enters into the tumor part Pa of the pancreas P. At this point, as explained above, the projection amount L5 of the bent blade distal end portion 72a from the opening portion 71c can be adjusted according to a movement amount of the blade slider 85 with respect to the needle tube turning operation portion 84.

FIG. 33 is a diagram showing an example of an ultrasound image displayed on the monitor 4. The ultrasound image USI obtained by the ultrasound probe 14 is displayed on the display screen of the monitor 4. An image 71bx of the needle tube distal end portion 71b and an image 72ax of the blade distal end portion 72a are clearly displayed on the ultrasound image USI by the ultrasound reflection machined portions provided on the surfaces of the needle tube distal end portion 71b and the blade distal end portion 72a. The image Pax of the tumor part Pa is also displayed on the ultrasound image USI.

Therefore, the doctor can grasp a position of the needle tube distal end portion 71b and a projection amount of the blade distal end portion 72a while viewing the ultrasound image USI.

The doctor can resect the tumor tissue of the tumor part Pa with the bending portion of the blade distal end portion 72a, which is the cutting portion, by locating the projected blade distal end portion 72a in a position where the inner portion of the tumor part Pa can be shaved off and turning the needle tube 71 around the axis while feeding a high frequency current to the blade 72 under the ultrasound guide.

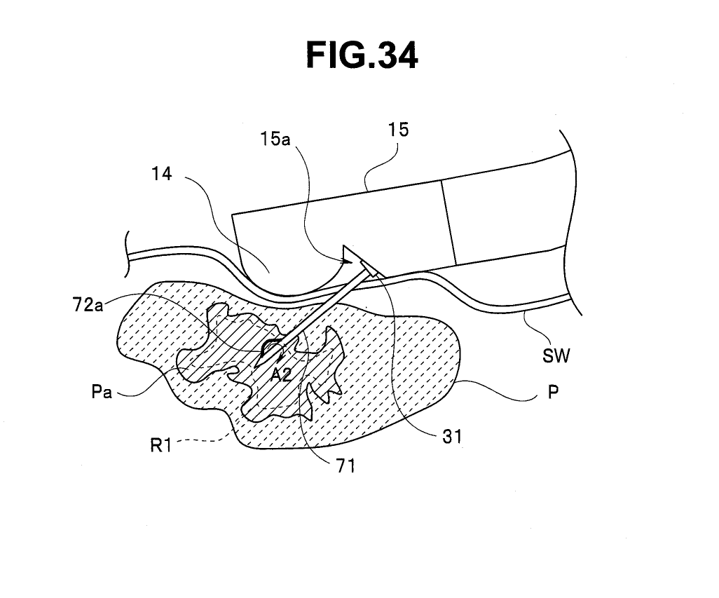

FIG. 34 is a diagram for explaining a movement of the blade distal end portion 72a. As shown in FIG. 34, the doctor moves the needle tube 71 in the inner portion of the tumor part Pa as indicated by a dotted line R1 while turning the needle tube 71 around the axis as indicated by an arrow A2. At this point, if the blade distal end portion 72a is turned as indicated by the arrow A2, since the high frequency current is flowing to the blade 72, the bending portion of the blade distal end portion 72a can shave off the tumor tissue and resect the tumor tissue into small tumor tissue pieces in the same manner as an electric knife. If the needle tube 71 is moved in the inner portion of the tumor part Pa as indicated by the dotted line R1 while turning the needle tube 71 around the axis, one tumor part Pa is decomposed into a large number of or a plurality of fine tumor tissue pieces. The doctor can determine, viewing the ultrasound image USI, a degree of the resection of the tumor tissue of the tumor part Pa, that is, to which degree the tumor tissue is resected.

It is preferable that the resection of the tumor tissue be performed to leave only a thin portion on the outer side surface of the tumor part Pa. That is, the doctor resects the tumor tissue pieces while viewing an ultrasound image until the entire tumor tissue is resected to leave a layer of an outer contour of the tumor part Pa without cutting a normal biological tissue of the pancreas P around the tumor part Pa. Note that a range to be resected may be only a desired region of the tumor part Pa.

Subsequently, the doctor injects saline or ethanol into the tumor part Pa through the needle tube distal end portion 71b under the ultrasound guide (S32).

The doctor pours saline or ethanol, which is a solvent for suspension, into the syringe main body of the syringe 25 in advance. After S31, as explained above, the doctor detaches the blade slider 85 of the puncture device 21A from the needle tube turning operation portion 84, pulls out the blade 72 from the needle tube 71, attaches the syringe 25 including the saline or the ethanol to the opening portion 84c of the proximal end portion of the needle tube turning operation portion 84, from which the blade slider 85 is pulled out, and pushes in the plunger 25b of the syringe 25 to thereby perform injection of the saline or the ethanol.

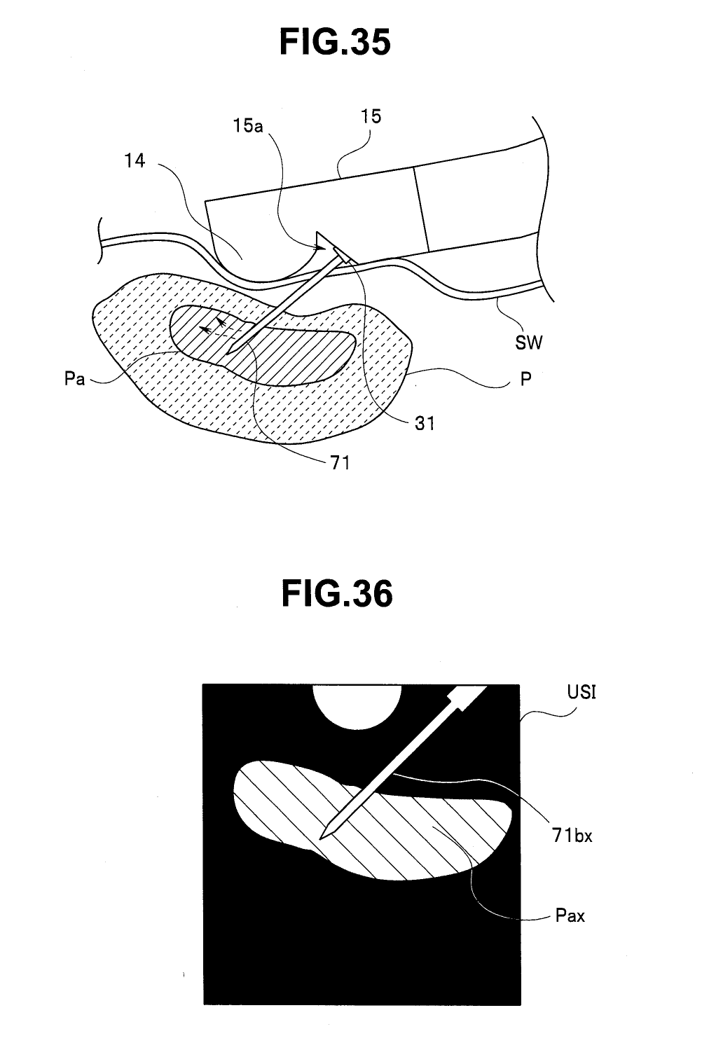

FIG. 35 is a diagram for explaining the injection of the saline or the ethanol. When the doctor injects the saline or the ethanol from the syringe 25, as indicated by a dotted line in FIG. 35, the saline or the ethanol is injected into an inner portion of the layer of the outer contour of the tumor part Pa from the opening portion 71c. Therefore, the tumor part Pa bulges and a volume of the tumor part Pa increases. The doctor can view a situation of the injection of the saline or the ethanol using an ultrasound image displayed on the monitor 4.

FIG. 36 is a diagram showing an example of the ultrasound image displayed on the monitor 4 when the saline or the ethanol is injected. As shown in FIG. 36, the image Pax of the bulged tumor part Pa is displayed on the monitor 4. Therefore, the doctor can inject the saline or the ethanol into the tumor part Pa by a desired amount while viewing the ultrasound image.

As explained above, the doctor introduces the solvent for suspension into the tumor inner portion from the distal end of the needle tube 71 arranged in the tumor inner portion, mixes the solvent for suspension with the resected tumor tissue in the tumor inner portion, and changes the tumor inner portion to fluid under the ultrasound observation.

The doctor collects the resected tumor tissue pieces together with the saline or the ethanol under the ultrasound guide (S33). The collection of the resected tumor tissue pieces is performed by the operation of the syringe 25.

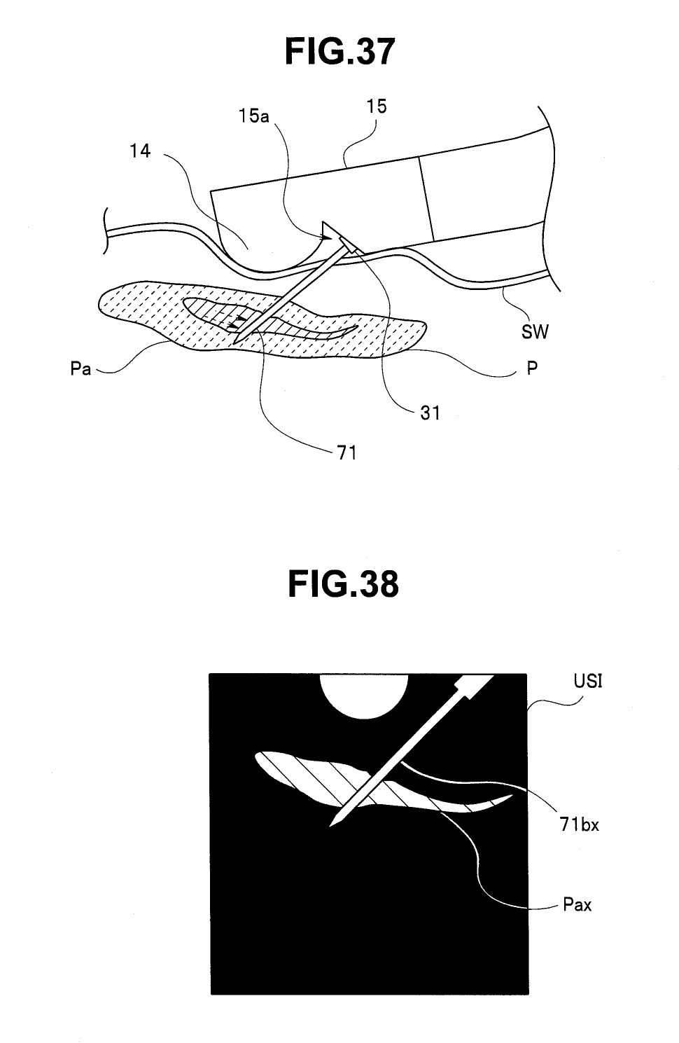

FIG. 37 is a diagram for explaining the collection of the resected tumor tissue pieces. When the doctor operates the plunger 25b of the syringe 25 to pull out the plunger 25b from the syringe main body, as indicated by a dotted line in FIG. 37, the resected tumor tissue pieces are sucked from the opening portion 71c together with the saline or the ethanol and the volume of the tumor part Pa decreases. That is, when the saline or the ethanol is injected into the tumor part Pa in S32, fine fragments of the resected tumor tissue are included in the saline or the ethanol. Therefore, when the resected tumor tissue is sucked by the syringe 25 together with the saline or the ethanol, the volume of the tumor part Pa decreases.

As explained above, after changing the tumor inner portion to the fluid, the doctor collects the tumor tissue, which is mixed with the solvent for suspension and softened, from the tumor inner portion using the needle tube 71.

The doctor can view a situation of the collection of the tumor tissue using the ultrasound image displayed on the monitor 4. FIG. 38 is a diagram showing an example of the ultrasound image displayed on the monitor 4 when the resected tumor tissue pieces are collected. As shown in FIG. 38, the image Pax of the reduced tumor part Pa is displayed on the monitor 4.

Further, the doctor pulls out the needle tube 71 from the inside of the tumor part Pa while feeding a high frequency current to the needle tube 71 (S34). Since the high frequency current is flowing to the needle tube 71, a biological tissue in contact with an outer surface of the needle tube 71 is burned by the high frequency current and dissemination can be prevented.

Therefore, according to the present embodiment, the same effects as those in the first embodiment are generated. That is, it is possible to reduce the volume of the tumor and reduce or eliminate compression of surrounding organs, blood vessels, and nerves. It is possible to perform a surgical operation such as excision of the tumor. Further, since the tumor tissue is sucked together with the ethanol after being dissolved, there is also an effect that tumor lysis syndromes due to remaining of a cancer cell or the like in the body are suppressed. A pain due to compression of nerves based on an increase in a tumor volume is also reduced and QOL (quality of life) is also improved.

Note that, in S32, under the ultrasound guide, after putting the needle tube distal end portion 71b through the tumor part Pa and injecting the saline or the ethanol, when the doctor introduces the ethanol, which is a solvent for suspension, into the inner portion of the tumor part Pa, mixes the ethanol with resected tumor cells, and changes the tumor inner portion to fluid, if the doctor turns the blade distal end portion 72a, it is possible to facilitate fluidization of the tumor inner portion. The doctor may perform the turning of the blade distal end portion 72a by hand or using a turning device incorporating a motor.

### (Modifications)

Note that, in the fourth embodiment, the configurations of the modification 1 explained in the first embodiment can also be applied. In the fourth embodiment, the same effects as those in the modification 1 of the first embodiment are generated.

### Fifth Embodiment

In the first to fourth embodiments, the tumor tissue is dissolved, broken up by the ultrasound vibration, or resected and the dissolved, broken-up, or resected tumor tissue is collected. However, in a fifth embodiment, a tumor tissue is vaporized by a laser and collected.

An apparatus configuration for realizing a manipulation in the present embodiment is substantially the same as the apparatus configuration in the first embodiment. The same components are denoted by the same reference signs and explanation of the components is omitted. Different components are mainly explained.

In the manipulation in the fifth embodiment, steps of a procedure same as in the first embodiment are denoted by the same reference signs and explanation of the steps is omitted.

### (Configuration)

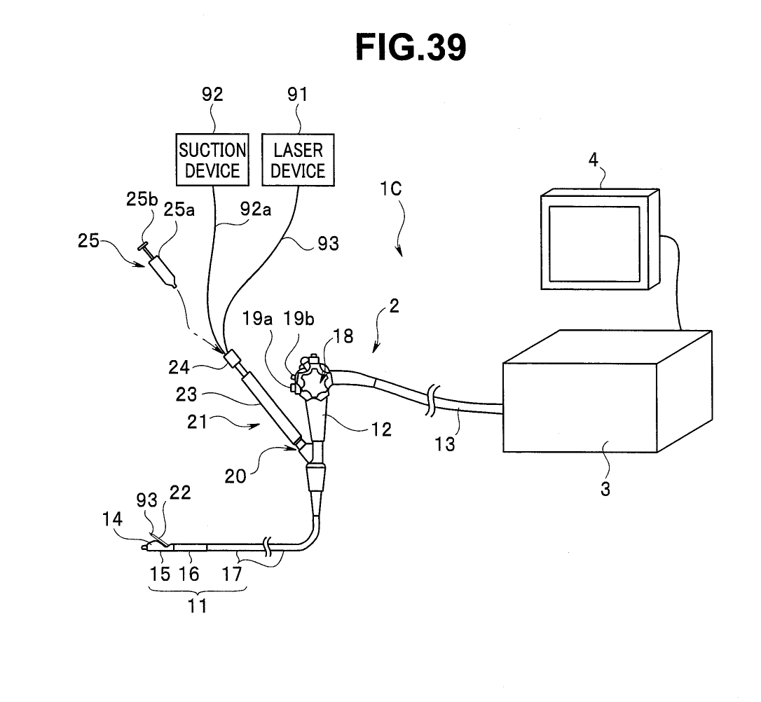

FIG. 39 is a configuration diagram showing a configuration of an ultrasound endoscope apparatus according to the present embodiment. In FIG. 39, components same as the components of the ultrasound endoscope apparatus 1 shown in FIG. 1 are denoted by the same reference signs and explanation of the components is omitted. An ultrasound endoscope apparatus 1C in the present embodiment includes a laser device 91, which is a vaporization tool for vaporizing a tumor inner portion, and a suction device 92.

The laser device 91 includes a laser light source on an inner portion. Laser light of the laser light source is emitted into a laser catheter 93. The laser catheter 93 is a probe that includes an optical fiber, makes the laser light from the laser device 91 incident from a proximal end portion of the optical fiber, and emits the laser light from a distal end portion of the laser catheter 93.

The laser device 91 is, for example, a carbon dioxide laser device or an infrared laser device. The laser light is, for example, light (CO₂ laser) having a wavelength of 10.6 μm and has power of a degree enough for cauterizing and vaporizing the tumor tissue.

The suction device 92 includes a suction pump on an inner portion. A suction tube 92a is connected to the suction device 92. A laser catheter 26a is inserted through the needle tube 22 from the proximal end portion of the needle tube 22. The suction tube 92a is connected to the operation slider 24. The operation slider 24 is configured such that the inside of the needle tube 22 is sucked through a suction tube 27a.

The laser catheter 93 is inserted through the needle tube 22. A gap is formed between an inner wall of the needle tube 22 and an outer surrounding surface of the laser catheter 93. The suction tube 92a communicates with the internal space of the needle tube 22.

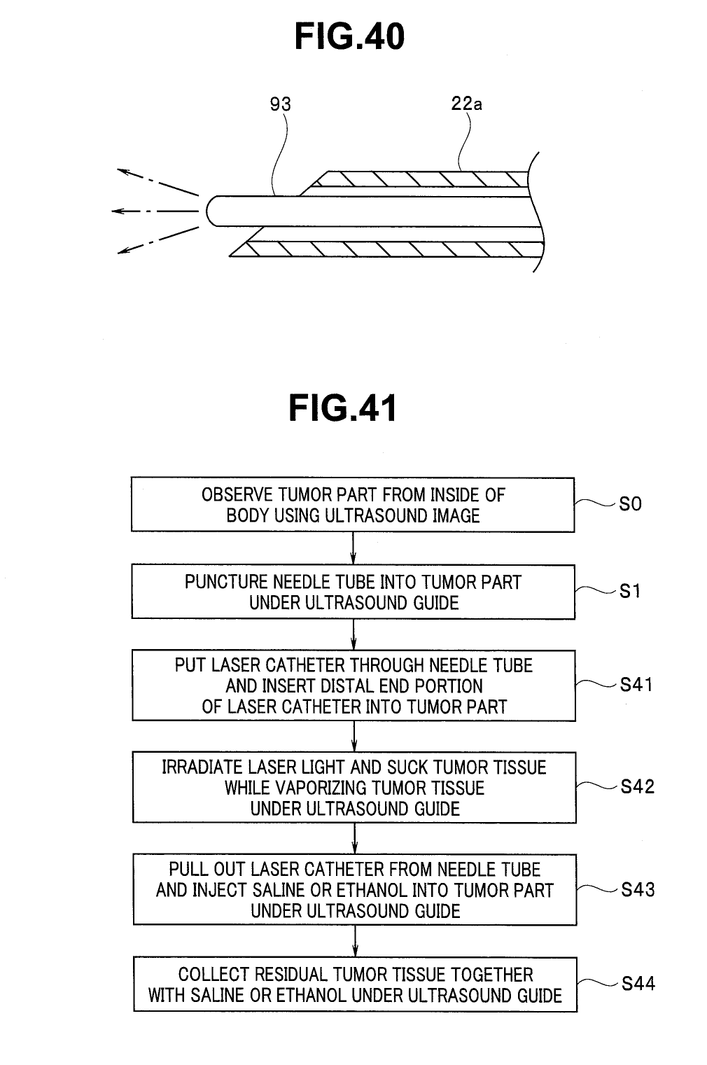

FIG. 40 is a schematic sectional view showing a configuration of a needle tube distal end portion through which the laser catheter 93 for radiating laser light on a tumor tissue and vaporizing the tumor tissue is inserted.

The needle tube 22 is inserted through the instrument channel of the ultrasound endoscope 2. Further, the laser catheter 93 is inserted through the needle tube 22. The laser light generated by the laser device 91 can be emitted from a distal end of the laser catheter 93 through the optical fiber.

There is a gap between an outer surface of the laser catheter 93 inserted through the needle tube 22 and an inner wall surface of the needle tube 22. The suction tube 92a communicates with the gap. As explained below, the suction device 92 can suck gas generated by vaporization via the gap using the suction tube 92a.

### (Manipulation)

FIG. 41 is a diagram for explaining a procedure of a manipulation in the fifth embodiment.

After S1, processing in S41 is performed. First, the doctor puts the laser catheter 93 through the needle tube 22 and inserts the distal end portion of the laser catheter 93 into the tumor part Pa (S41). The doctor can confirm, by viewing the ultrasound image, that the distal end portion of the laser catheter 93 is arranged in a desired position.

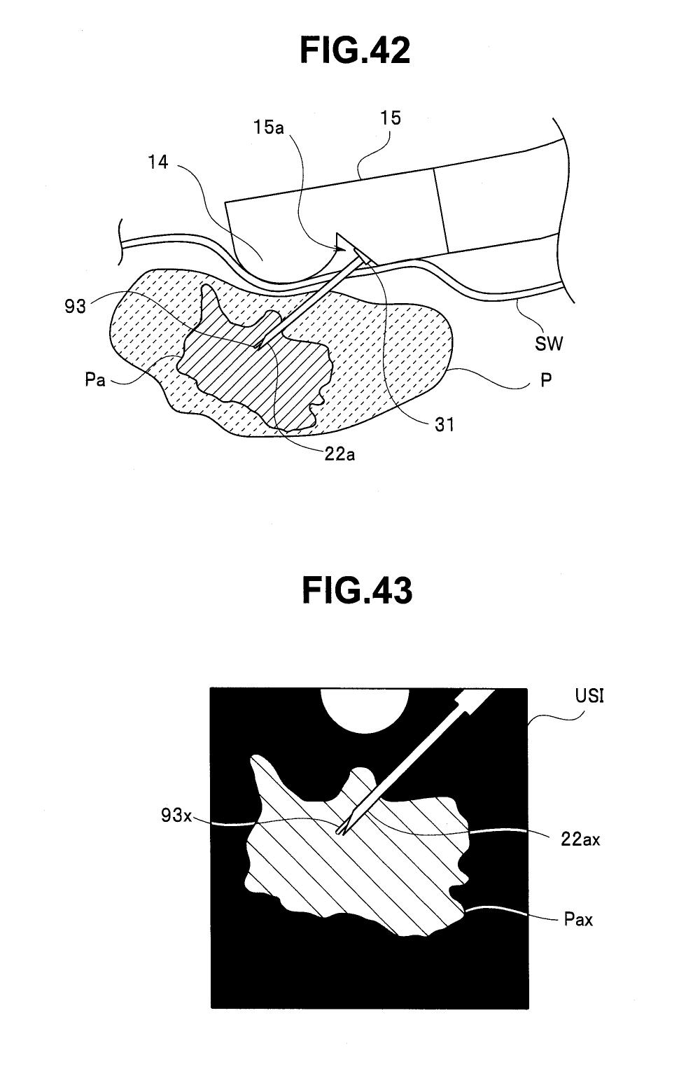

FIG. 42 is a diagram for explaining a projecting state of the needle tube distal end portion 22a of the needle tube 22. As shown in FIG. 42, the distal end portion of the laser catheter 93 is inserted into the tumor part Pa.

FIG. 43 is a diagram showing an example of an ultrasound image displayed on the monitor 4 corresponding to FIG. 42. The image 22ax of the needle tube distal end portion 22a, an image 93x of the laser catheter 93, and the image Pax of the tumor part Pa are also displayed on the ultrasound image USI.

Subsequently, the doctor sucks the tumor tissue while radiating laser light on the tumor tissue and vaporizing the tumor tissue using the laser catheter 93 inserted through the needle tube 22 under the ultrasound guide (S42). That is, the doctor vaporizes the tumor inner portion by radiating the laser light on the tumor tissue under the ultrasound observation by the ultrasound observation device 3.

Specifically, the doctor drives the laser device 91, which is the vaporization tool for vaporizing a tumor inner portion, emits the laser light from the distal end of the laser catheter 93, and drives the suction device 92 as well under the ultrasound guide. As a result, the tumor tissue is heated and vaporized by the laser light. Gas generated by the vaporization is sucked by the suction device 92.

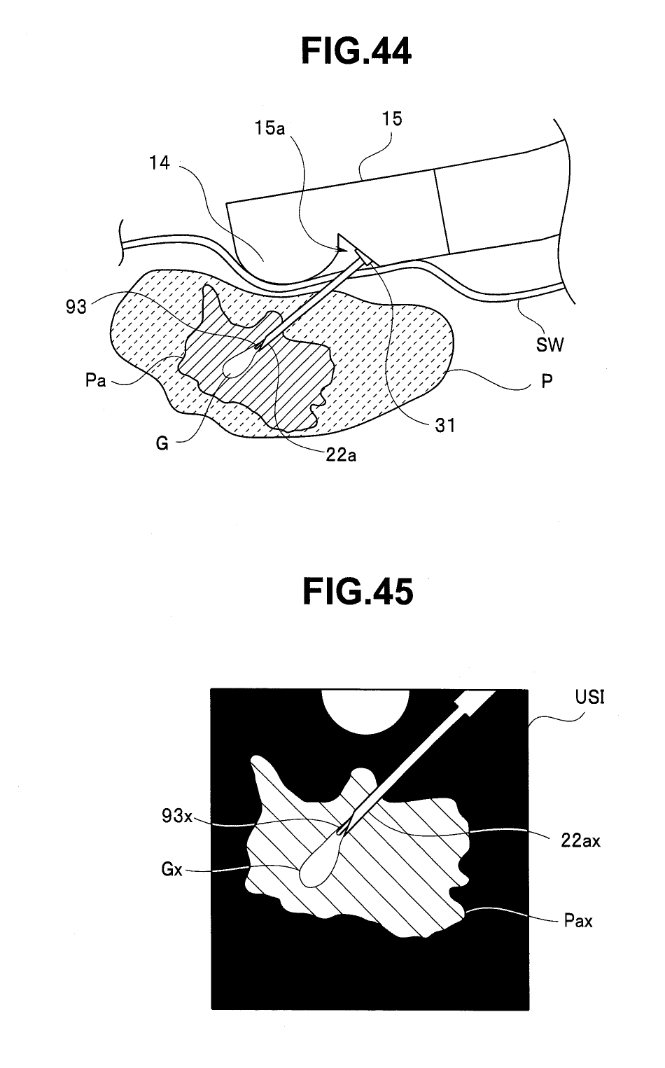

FIG. 44 is a diagram for explaining a state in which the laser light is radiated on the tumor tissue and the tumor tissue is vaporized. FIG. 45 is a diagram showing an example of an ultrasound image displayed on the monitor 4 in the state shown in FIG. 44.

When the laser light is radiated on the tumor tissue, the tumor tissue vaporizes and changes to gas. FIG. 44 shows that the tumor tissue changes to gas G. In the ultrasound image USI shown in FIG. 45, an image Gx of the gas G in the tumor part Pa is also rendered.

For example, the doctor radiates the laser light and vaporizes the tumor tissue in a desired region while viewing the ultrasound image displayed on the monitor 4. The laser device 91 and the suction device 92 are driven by, for example, operating operation devices such as foot switches connected to the respective devices.

A change of a radiating direction of the laser light can be performed by bending the bending portion 16 of the insertion portion 11.

For example, the doctor can determine, by viewing the ultrasound image, a degree of the vaporization of the tumor tissue, that is, to which degree the tumor tissue is vaporized.

The doctor performs the vaporization while viewing the ultrasound image until the entire tumor tissue or the desired region is vaporized.

Subsequently, the doctor pulls out the laser catheter 93 from the needle tube 22 and injects saline or ethanol into the tumor part Pa through the needle tube 22 under the ultrasound guide (S43).

When the vaporization of the tumor tissue ends in S42, the doctor detaches the laser catheter 93 and the suction tube 92a from the operation slider 24 and connects the syringe 25 to the operation slider 24. The doctor can inject the saline or the ethanol, which is a solvent for suspension, into the needle tube 22 and inject the saline or the ethanol into the tumor part by pouring the saline or the ethanol into the syringe main body 25a and pushing in the plunger 25b.

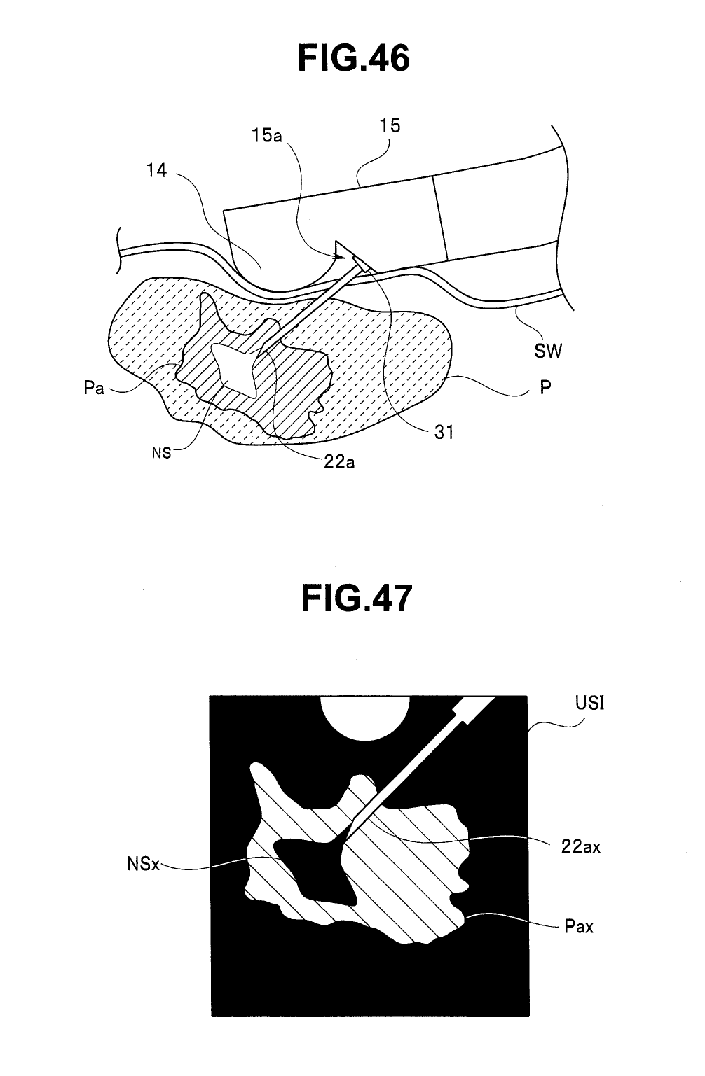

FIG. 46 is a diagram for explaining a state at the time when the saline or the ethanol is injected into the tumor part Pa. FIG. 47 is a diagram showing an example of an ultrasound image displayed on the monitor 4 in the state shown in FIG. 46.

In the ultrasound image USI shown in FIG. 47, an image NSx of the saline or ethanol NS in the tumor part Pa is rendered in black.

As explained above, the doctor introduces the solvent for suspension into the tumor inner portion from the distal end of the needle tube 22 arranged in the tumor inner portion, mixes the solvent for suspension with the vaporized tumor tissue in the tumor inner portion, and changes the tumor inner portion to fluid under the ultrasound observation.

The doctor collects the tumor tissue remaining after the vaporization by the laser beam together with the saline or the ethanol under the ultrasound guide (S44).

When the saline or the ethanol is injected in S43, the tumor tissue remains in the saline or the ethanol. Therefore, when the remaining tumor tissue is sucked together with the saline or the ethanol, it is possible to reduce a volume of a portion where the tumor tissue is present.

That is, since the tumor tissue is vaporized by the laser light and the remaining tumor tissue is sucked, a volume of the tumor tissue decreases.

As explained above, after changing the tumor inner portion to the fluid, the doctor collects the tumor tissue, which is mixed with the solvent for suspension and softened, from the tumor inner portion using the needle tube 22.

As a result, it is possible to reduce the volume of the tumor and reduce or eliminate compression of surrounding organs, blood vessels, and nerves. It is possible to excise the tumor. Further, since the remaining tumor tissue is sucked, there is also an effect that tumor lysis syndromes due to remaining of a cancer cell or the like in the body are suppressed. A pain due to compression of nerves based on an increase in a tumor volume is also reduced and QOL (quality of life) is also improved.

Note that, in the fifth embodiment explained above, the needle tube 22 is used. However, the doctor may use a double lumen tube, put the laser catheter 93 through one lumen, use the other lumen as a suction channel, and, while vaporization of the tumor tissue is performed by the laser catheter 93, perform suction of gas generated by the vaporization.

As explained above, with the tumor reductive procedure under the ultrasound observation in the respective embodiments and the respective modifications, it is possible to reduce the volume of the tumor and reduce or eliminate compression of surrounding organs, blood vessels, and nerves. It is possible to perform a surgical operation such as excision of the tumor. A clinical condition such as a pain due to compression based on hypertrophy of a tumor is also reduced. Therefore, a number of times of treatment for reducing the pain or the like also decreases. As a result, QOL (quality of life) of a patient is also improved.

Further, the tumor tissue is sucked together with the ethanol after being dissolved. Therefore, there is also an effect that tumor lysis syndromes due to remaining of a cancer cell or the like in the body are suppressed.

### (Modifications)

Note that, in the fifth embodiment, the configuration of the modification 1 explained in the first embodiment can also be applied. In the fifth embodiment, the same effects as those in the modification 1 of the first embodiment are generated.

Next, modifications related to all the embodiments explained above are explained.

### (Modification 5)

When the tumor inner portion is fluidized, the ultrasound probe 14 may be driven for a predetermined time once or more at a frequency determined in advance to agitate the solvent for suspension in the tumor part Pa. The solvent for suspension is sufficiently spread to the inside of the tumor part Pa and further fluidization of the tumor inner portion is performed according to the agitation by the ultrasound vibration.

For example, as disclosed in Japanese Patent No. 5019997, a C-MUT or P-MUT technology may be used for the ultrasound probe 14 of the endoscope to enable the ultrasound probe 14 to output ultrasound vibrations at a plurality of frequencies. During the agitation, the ultrasound probe 14 may be driven at a frequency suitable for the agitation to output ultrasound.

Further, the needle tube may be warmed by feeding a predetermined electric current to the needle tube. The solvent for suspension is also sufficiently spread to the inside of the tumor part Pa by the warming.

Furthermore, the warming may be performed from a body surface.

### (Modification 6)

In the respective embodiments explained above, the pancreatic cancer is explained as an example of the tumor. However, the tumor is not limited to the pancreatic cancer. The manipulations in the respective embodiments explained above can also be applied when tumors of other organs (e.g., liver) are reduced.

### (Modification 7)

In the respective embodiments explained above, the endoscope insertion portion is inserted, that is, introduced into the body from the mouth of the patient. However, the endoscope insertion portion may be introduced into the body from parts other than the mouth. That is, the endoscope insertion portion may be introduced into the body per os, per nasal, or per anal.

### (Modification 8)

Furthermore, the ethanol, the plasmin antagonist, and the saline are explained as the examples of the solvent for suspension. However, the solvent for suspension may be other kinds of alcohol such as isopropanol (isopropyl alcohol) or hydrogen peroxide water, dispase, collagenase, thermolysin, pronase, hyaluroninase, pancreatin, or elastase.

### (Modification 9)

In the respective embodiments explained above, the ultrasound observation is performed from the inside of the body. However, the ultrasound observation may be performed from the outside of the body. That is, in the respective embodiments explained above, the ultrasound observation is performed by obtaining the ultrasound image of the tumor part using the ultrasound probe provided in the endoscope insertion portion 11 inserted into the body. However, ultrasound may be radiated from a body surface of a patient to obtain an ultrasound image.

### (Modification 10)

Further, in the fourth embodiment, the dissemination prevention is attained by pulling out the needle tube from the inside of the tumor part while feeding the high frequency current to the needle tube. However, the doctor may perform the dissemination prevention by pulling out the needle tube while ejecting ethanol from a tip of the needle tube or by pulling out the needle tube while heating the needle tube distal end portion with laser light or a heat probe.

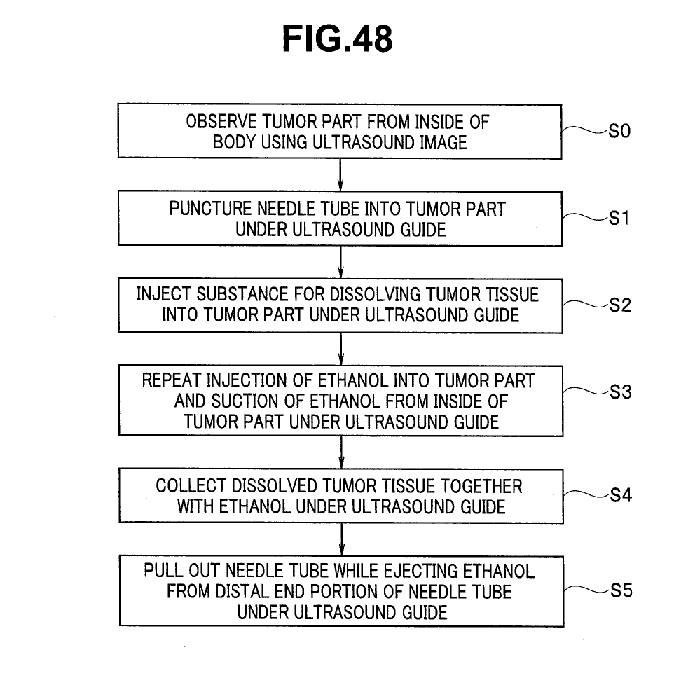

FIG. 48 is a flowchart explaining a flow of a manipulation including the processing for the dissemination prevention in the first embodiment. As shown in FIG. 48, after S4, the doctor attaches the syringe, into which ethanol is poured, to the proximal end portion of the operation slider 24 and pulls out the needle tube 22 while ejecting the ethanol from the distal end portion of the needle tube 22 under the ultrasound guide (S5).

Processing in S5 can be applied to not only the manipulation in the first embodiment but also the manipulations in the second, third, and fifth embodiments. Further, in the fourth embodiment, the processing in S5 can be performed instead of S34. Therefore, the processing in S5 can also be applied to the manipulation in the fourth embodiment.

Furthermore, the doctor may perform the dissemination prevention by pulling out the needle tube while feeding an electric current to the needle tube or feeding warm water into the needle tube to heat the needle tube to a predetermined temperature (e.g., 42 degrees).

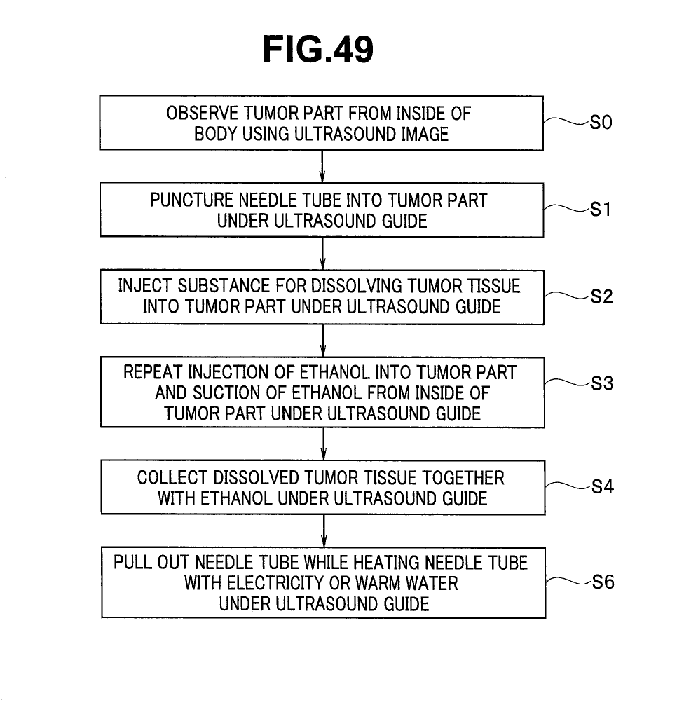

FIG. 49 is a flowchart showing a flow of a manipulation of the processing of the other dissemination prevention in the first embodiment. As shown in FIG. 49, after S4, the doctor pulls out the needle tube under the ultrasound guide while applying a voltage for warming the needle tube to a predetermined temperature to the needle tube or feeding warm water having temperature for warming the needle tube to the predetermined temperature into the needle tube (S6).

Processing in S6 can be applied to not only the manipulation in the first embodiment but also the manipulations in the second, third, and fifth embodiments. Further, in the fourth embodiment, the processing in S6 can be performed instead of S34. Therefore, the processing in S6 can also be applied to the manipulation in the fourth embodiment.

Further, the doctor may pull out the needle tube while emitting laser light of a degree for warming the needle tube distal end portion 22a to a predetermined temperature (e.g., 42 degrees) from the distal end portion of the laser probe 32 shown in FIG. 11 to heat the needle tube distal end portion 22a.

Note that the doctor may pull out the needle tube while inserting the heat probe 33 shown in FIG. 11 into the needle tube rather than the laser probe 32 and heating a heater to a degree for warming the needle tube distal end portion 22a to the predetermined temperature (e.g., 42 degrees).

### (Modification 11)

Further, in the respective embodiments explained above, the tumor, which is mixed with the solvent for suspension and softened, is collected from the inner portion of the tumor part using the needle tube. However, the tumor may be discharged to a digestive tract or the outside of the body using a drainage tube.

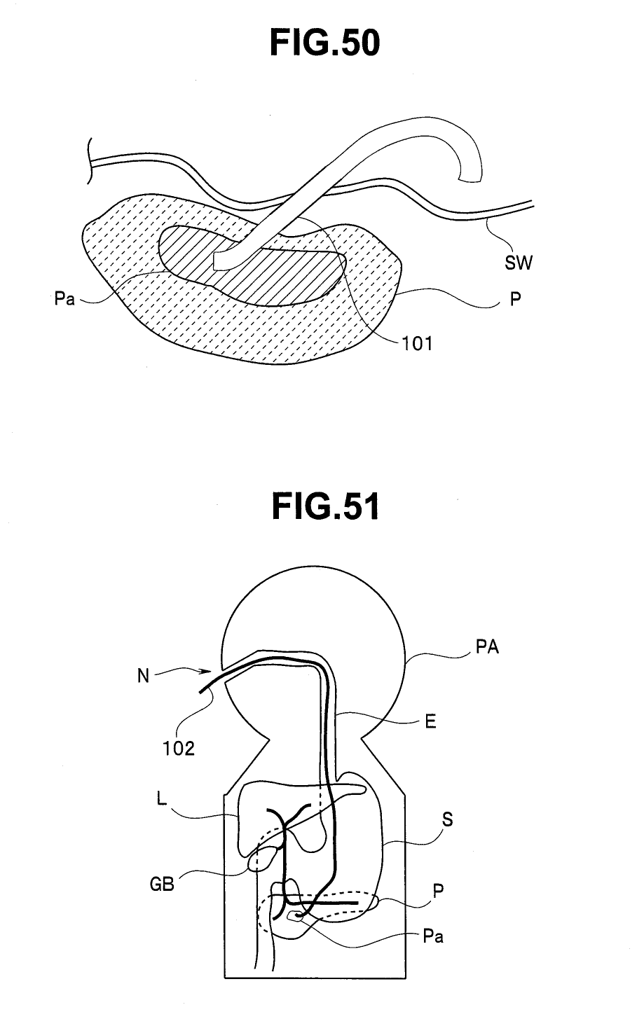

FIG. 50 is a diagram for explaining a method of discharging a tumor, which is mixed with a solvent for suspension and softened, from an inner portion of a tumor part to the stomach S, which is a digestive tract, using an internal biliary drainage tube 101. The internal biliary drainage tube 101 is a tube for connecting a tumor part and a digestive tract per digestive tract and indwelled the tumor inner portion a body. The tumor, which is mixed with the solvent for suspension and softened, is naturally discharged into the digestive tract via the internal biliary drainage tube 101. Note that the internal biliary drainage tube 101 connects the tumor part Pa and the stomach S. However, the internal biliary drainage tube 101 may connect the tumor part Pa and a duodenum or the like.

FIG. 51 is a diagram for explaining a method of discharging a tumor, which is mixed with a solvent for suspension and softened, to an outside of a body from an inner portion of a tumor part using an external biliary drainage tube 102. The external biliary drainage tube 102 connects a tumor part and an outside world per nasal. One end of the external biliary drainage tube 102 is indwelled in the body and the other end thereof is extended to the outside of the body. The tumor, which is mixed with the solvent for suspension and softened, is naturally discharged to the outside of the body via the external biliary drainage tube 102 inserted from a nose N. Note that an inner portion of the tumor part may be forcibly refluxed from the outside world making use of the external biliary drainage tube 102.

Note that, in the example explained above, the internal biliary drainage tube 101 or the external biliary drainage tube 102 is used. However, both of the internal biliary drainage tube 101 and the external biliary drainage tube 102 may be used to naturally discharge or forcibly discharge the softened tumor.

In this way, the tumor tissue may be discharged using the drainage tube without being collected.

The present invention is not limited to the respective embodiments and the respective modifications explained above. Various changes, alterations, and the like are possible in a range in which the gist of the present invention is not changed.

What is claimed is:

1. A tumor reductive procedure under ultrasound observation comprising:
observing a tissue from an inside or an outside of a body with an ultrasound observation device;
dissolving, breaking up, resecting, or vaporizing a tumor inner portion under ultrasound observation by the ultrasound observation device;
introducing a solvent for suspension into the tumor inner portion from a distal end of a needle tube arranged in the tumor inner portion, mixing the solvent for suspension with a tumor tissue of the dissolved, broken-up, resected, or vaporized tumor inner portion, and changing the tumor inner portion to a fluid under the ultrasound observation; and
collecting the tumor tissue, which is mixed with the solvent for suspension and softened, from the tumor inner portion using the needle tube or another duct after changing the tumor to the fluid.

2. The tumor reductive procedure under ultrasound observation according to claim 1, wherein the solvent for suspension is ethanol, plasmin antagonist, or saline.

3. The tumor reductive procedure under ultrasound observation according to claim 1, wherein the tumor inner portion is changed to the fluid by mixing the dissolved, broken-up, resected, or vaporized tumor tissue with the solvent for suspension while agitating, warming, or refluxing the tumor tissue.

4. The tumor reductive procedure under ultrasound observation according to claim 1, wherein a distal end of the needle tube is introduced into the tumor inner portion and the tumor inner portion is dissolved by injecting a substance for dissolving the tumor inner portion through the needle tube under the ultrasound observation.

5. The tumor reductive procedure under ultrasound observation according to claim 4, wherein the solvent for suspension is a substance for neutralizing the substance for dissolving the tumor inner portion.

6. The tumor reductive procedure under ultrasound observation according to claim 1, wherein the breaking-up of the tumor inner portion is performed using a breaking-up tool for breaking up the tumor inner portion.

7. The tumor reductive procedure under ultrasound observation according to claim 6, wherein the breaking-up tool is a probe including an ultrasound transducer.

8. The tumor reductive procedure under ultrasound observation according to claim 1, wherein the resection of the tumor inner portion is performed using a resecting tool for resecting the tumor inner portion.

9. The tumor reductive procedure under ultrasound observation according to claim 8, wherein the resecting tool is a bendable blade.

10. The tumor reductive procedure under ultrasound observation according to claim 1, wherein the vaporization of the tumor inner portion is performed using a vaporizing tool for vaporizing the tumor inner portion.

11. The tumor reductive procedure under ultrasound observation according to claim 10, wherein the vaporizing tool is a probe capable of emitting laser light.

12. The tumor reductive procedure under ultrasound observation according to claim 1, further comprising pulling out the needle tube or the other duct while ejecting ethanol from a distal end portion of the needle tube or the other duct.

13. The tumor reductive procedure under ultrasound observation according to claim 1, further comprising pulling out the needle tube or the other duct while heating a distal end portion of the needle tube or the other duct.

14. The tumor reductive procedure under ultrasound observation according to claim 1, wherein the other duct is a drainage tube communicating with the inside of the body or the outside of the body.

15. The tumor reductive procedure under ultrasound observation according to claim 1, wherein
the ultrasound observation device is an ultrasound endo scope including an instrument channel,
the ultrasound endoscope is introduced into the body per os, per nasal, or per anus, and
the needle tube or the other duct is inserted through the instrument channel and introduced into the tumor inner portion.
